(19) United States
(12) Patent Application Publication
(10) Pub. No.: US 2022/0240845 A1
(43) Pub. Date: Aug. 4, 2022

(54) SYSTEM, METHOD AND COMPUTER READABLE MEDIUM FOR RAPIDLY PREDICTING CARDIAC RESPONSE TO A HEART CONDITION AND TREATMENT STRATEGY

(71) Applicant: University of Virginia Patent Foundation, Charlottesville, VA (US)

(72) Inventors: Jeffrey W. Holmes, Charlottesville, VA (US); Colleen Witzenburg, Madison, WI (US); Pim Oomen, Charlottesville, VA (US); Kenneth C. Bilchick, Charlottesville, VA (US)

(73) Assignee: University of Virginia Patent Foundation, Charlottesville, VA (US)

(21) Appl. No.: 17/436,378

(22) PCT Filed: Mar. 11, 2020
(86) PCT No.: PCT/US2020/022057

§ 371 Date: Sep. 3, 2021

Related U.S. Application Data
(60) Provisional application No. 62/817,644, filed on Mar. 13, 2019.

(51) Int. Cl. A61B 5/00; G16H 50/30; G16H 20/00

(52) U.S. Cl. CPC A61B 5/4833; G16H 20/00; G16H 50/30; A61B 5/7275

(57) ABSTRACT

A method and system for rapidly predicting cardiac response to a heart condition and treatment strategy of a patient. Predictions are accomplished using a compartmental model that includes systemic and pulmonary circulations represented as a system of resistors and capacitors together with chambers of the heart represented as simple geometric shapes such as spheres or assemblies of spheres or other analytic equations that relate pressure and volume to stress and strain. The model is calibrated using disease-specific data sets for a specific heart condition. Simple geometry modeling allows for rapid use such that hemodynamic parameters may be tuned for optimal accuracy. Treatment strategies may be modified and re-simulated in real time if necessary to achieve a more optimal outcome and treatment.

### RELATED APPLICATIONS

The present application is a national stage filing of International Application No. PCT/US2020/022057, filed Mar. 11, 2020, which claims benefit of priority under 35 U.S.0 § 119 (e) from U.S. Provisional Application Ser. No 62/817,644, filed Mar. 13, 2019, entitled “System, Method and Computer Readable Medium for Predicting the Time Course of Ventricular Dilation and Thickening”; the disclosures of which are hereby incorporated by reference herein in their entirety.

### STATEMENT OF GOVERNMENT INTEREST

This invention was made with government support under Grant No. HL127654, awarded by The National Institutes of Health. The government has certain rights in the invention.

### FIELD OF INVENTION

The present disclosure relates generally to modeling cardiac responses to heart conditions and simulated treatments. More particularly, rapidly modeling a disease-specific cardiac response to a heart condition and predicting cardiac response to a patient-specific treatment strategy based on tuned hemodynamic parameters or other parameters.

### BACKGROUND

Pathologies such as congenital heart disease, hypertension, valvular disease, and myocardial infarction cause the heart to grow and remodel. In many cases, this remodeling contributes to the deterioration of cardiac function and the development of heart failure [See Savinova, O. V., & Gerdes, A. M. (2012). Myocyte changes in heart failure. Heart Failure Clinics, 8(1), 1-6] [See O'Gara, P. T., Kushner, F. G., Ascheim, D. D., Casey, D. E., Chung, M. K., De Lemos, J. A., et al. (2013). 2013 ACCF/AHA guideline for the management of stelevation myocardial infarction. Circulation, 127(4), e362-425]. Thus, increases in ventricular mass, diameter, and wall thickness [See Yancy, C. W., Jessup, M., Bozkurt, B., Butler, J., Casey, D. E., Drazner, M. H., et al. (2013). 2013 ACCF/AHA Guideline for the Management of Heart Failure. Circulation, 128(16), 1810-1852] [See Gardin, J. M., Mcclelland, R., Kitzman, D., Lima, J. A. C., Bommer, W., Klopfenstein, H. S., et al. (2001). M-mode echocardiographic predictors of six- to seven-year incidence of coronary heart disease, stroke, congestive heart failure, and mortality in an elderly cohort (The Cardiovascular Health Study). The American Journal of Cardiology, 87(9), 1051-1057] [See Aurigemma, G. P., Gottdiener, J. S., Shemanski, L., Gardin, J., & Kitzman, D. (2001). Predictive value of systolic and diastolic function for incident congestive heart failure in the elderly: the cardiovascular health study. Journal of the American College of Cardiology, 37(4), 1042-1048] [See Nishimura, R. A., Otto, C. M., Bonow, R. O., Carabello, B. A., Erwin, J. P., Guyton, R. A., et al. (2014). 2014AHA/ACC guideline for the management of patients with valvular heart disease: executive summary. Circulation, 129(23), 2440-2492] have all been associated with poor clinical prognosis.

Since remodeling of the ventricle is often progressive, the most pertinent clinical questions surrounding disorders that induce remodeling tend to be prognostic. Clinicians often face difficult decisions not only about the type of treatment but also about the timing, constantly weighing the trade-offs of delaying vs. performing a given repair at a specific time in an individual patient. For example, patients with mitral or aortic insufficiency are at increased risk for heart failure. Surgical repair or replacement of the valve can arrest or even reverse ventricular remodeling and preserve or restore function [See Nishimura, R. A., Otto, C. M., Bonow, R. O., Carabello, B. A., Erwin, J. P., Guyton, R. A., et al. (2014). 2014AHA/ACC guideline for the management of patients with valvular heart disease: executive summary. Circulation, 129(23), 2440-2492]. Because these benefits diminish with disease progression, early intervention has been associated with lower onset of heart failure rates and higher long-term survival [See Bonow, R. O., Carabello, B. A., Chatterjee, K., de Leon, A. C., Faxon, D. P., Freed, M. D., et al. (2008). 2008. Focused Update Incorporated Into the ACC/AHA 2006 Guidelines for the Management of Patients With Valvular. Heart Disease. Circulation, 118(15), e523-e661] [See Suri, R. M., Vanoverschelde, J., Grigioni, F., Schaff, H. V., Tribouilloy, C., Avierinos, J., et al. (2013). Association between early surgical intervention vs watchful waiting and outcomes for mitral regurgitation due to flail mitral valve leaflets. The Journal of the American Medical Association, 310(6), 609-616] On the other hand, not all patients require intervention, and in some the risks and complications (such as endocarditis, atrial or even ventricular fibrillation, embolic/bleeding events, and eventual deterioration of a prosthetic valve) will outweigh the benefits. Similar dilemmas exist for clinicians treating patients with congenital heart abnormalities. For example, high levels of pulmonary vascular resistance and low birth weight increase the risks associated with early surgical intervention for infants with single ventricles, but prolonged overloading of their single ventricle gradually reduces the efficacy of surgery [See Feinstein, J. A., Benson, D. W., Dubin, A. M., Cohen, M. S., Maxey, D. M., Mahle, W. T., et al. (2012). Hypoplastic left heart syndrome: current considerations and expectations. Journal of the American College of Cardiology, 59(1 SUPPL), S1-S42] Often, infants with single ventricle abnormalities develop heart failure so rapidly that surgery is no longer advised. Thus, in many situations, the ability to reliably predict growth and remodeling of the heart in individual patients could be a valuable tool for clinicians in anticipatory management, allowing them to predict both whether and when the benefits of intervention outweigh the risks.

Computational models are promising tools for integrating patient-specific data to generate meaningful predictions. In the past two decades, there has been considerable progress in the development of models capable of predicting cardiac remodeling in the setting of hypertension, valvular disease, and other pathologies. These models typically rely on mathematical equations, termed “growth laws,” that predict remodeling based on changes in one or more local mechanical inputs. These laws are grounded in experimental observations that hemodyanmic perturbations known to cause myocardial hypertrophy also alter the stress and strain within the myocardium. Although this approach is broadly consistent with experimental evidence that cardiac myocytes can sense and respond to changes in mechanics, the models are typically phenomenologic, derived from fitting data rather than attempting to represent the underlying myocyte biology. In the US alone, over 5 million patients suffer from heart failure, a number that is projected to exceed 8 million by 2030 [See Mozaffarian et al., Circulation, 113: e38-e360, 2016] Heart failure increases the likelihood of conduction abnormalities such as left bundle branch block (LBBB), which causes uncoordinated contraction and dilation of the left ventricle, leading to reduced pump efficiency [See Vernooy et al., Eur Heart J, 26(1): 91-98, 2005] [See Bilchick et al., J Am Coll Cardiol, 63(16): 1657-1666, 2014]

In the last two decades, cardiac resynchronization therapy (CRT) has emerged as a revolutionary therapy for patients with heart failure and LBBB. A CRT pacing device can restore coordinated contraction of the heart by electrically stimulating multiple locations at appropriate times. When it works, CRT can stop and even reverse the progression of heart failure, reducing the ventricle size and improving pump function. Many patients experience favorable LV remodeling and clinical improvement with CRT [See St. John Sutton et al., Circulation, 107(15): 1985-1990, 2003], but 30-50% do not have the desired response to this therapy [See Brignole et al., Eur. Heart J., 34(29): 2281-2329, 2013.] One of the greatest strengths of CRT is that it can be customized to individual patients, offering the potential to improve patient response rates [See Bilchick et al., J Am Coll Cardiol, 63(16): 1657-1666, 2014]. Yet this also presents a dilemma: there are far too many possible lead locations and pacing settings to test directly during the implantation surgery.

Computational models can address this challenge by rapidly screening many pacing options before the CRT device implantation takes place. These models could in theory be used to pre-identify pacing locations which lead not only to the best electrical and mechanical synchrony but also to the greatest long-term reduction in ventricular volume.

Long-term remodeling from stimulation at any one pacing location varies significantly from patient to patient, and is significantly influenced by tissue characteristics at the LV pacing site, including mechanical activation and presence of scar [See Bilchick et al., J. Am. Coll. Cardiol., 63(16): 1657-1666, 2014]. These observations suggest that optimizing pacing sites for individual patients could improve outcomes; however, there are too many possible pacing sites and settings to test in real-time during the implantation procedure. Therefore, there is a critical need for computational models that can predict outcomes (change of LV size) for various possible lead locations preoperatively. Several finite element models have been published that are capable of predicting cardiac growth, including in response to LBBB [See Kerckhoffs et al., Europace, 14(5): v65-v72, 2012] and CRT [See Arumugamet al., Sci. Rep., 9: 2019]; however, these models are computationally expensive, making them impractical for routine clinical use. The present inventor developed a fast compartmental model that can predict cardiac growth following volume and pressure overload [See Witzenburg et al., J Cardiovasc Trans Res, 11(2): 109-122, 2018], as well as LBBB [See Oomen et al., SB3C 2019]. The present inventor extends this framework, among other things, with a fast electrical model to predict cardiac growth following CRT. For example, previous the finite-element models were required to run for 3 weeks on a cluster with 12 6-core processors to simulate 4 weeks of growth [See Kerckhoffs et al., Europace, 14(5): v65-v72, 2012] [See Witzenburg et al., J Cardiovasc Trans Res, 11(2): 109-122, 2018], whereas in an embodiment the present inventor's model simulated 16 weeks of cardiac growth in just under two minutes on a laptop computer, making it suitable for routine clinical use.

The ability to reliably predict growth and remodeling of the heart in individual patients could have widespread clinical applications. Ideally, growth models intended for clinical use should capture ventricular growth across a range of different hemodyanmic conditions yet be fast enough to construct and run that they can support decision-making on the time scale of a hospital admission or even office visit. The compartmental growth model presented here was able to capture both the time course and distinct patterns of hypertrophy following aortic constriction, mitral valve regurgitation, and myocardial infarction for up to 3 months with simulation times of just a few minutes, suggesting promise for future clinical application.

### SUMMARY OF ASPECTS OF EXEMPLARY EMBODIMENTS OF THE INVENTION

An aspect of an embodiment includes a system, method and computer readable medium for providing, among other things, modeling cardiac responses to heart conditions and simulated treatments. An aspect of an embodiment includes a system, method and computer readable medium for providing, among other things, rapidly modeling a disease-specific cardiac response to a heart condition and predicting cardiac response to a patient-specific treatment strategy based on tuned hemodynamic parameters or other parameters.

An aspect an embodiment of the present invention includes representing chambers of the heart using simpler geometry or more efficient geometry compared to current approaches. Moreover, current approaches are subjected to limitations based on, but not limited thereto, representing chambers of the heart as complex geometric attributes of the heart to achieve accuracy. An aspect an embodiment of the present invention includes tuning hemodynamic parameters or other parameters to achieve more accurate inputs for predicting cardiac response to a heart condition and treatment strategy.

Phenomenologic growth laws use changes in mechanics to predict the rate of growth and remodeling. Thus, accurate predictions of the time course of growth require accurate matching of the hemodyanmic perturbations that precipitate it. Thus, the present inventor expects that patient-specific tuning of hemodynamic parameters will be an essential step in clinical applications of growth models such as the one presented here. While some of the hemodyanmic parameters in the model presented here—such as heart rate and systemic vascular resistance—can be directly measured or easily estimated for each subject, other parameters—such as the stressed blood volume—are very difficult to measure and therefore must be estimated from other measurable data through some sort of optimization routine. In a study of the present inventor study, the present inventor generated an algorithm to automatically tune model parameters for the left ventricle and circulatory system to match measured control and acute hemodynamics by minimizing the error in reported control and acute levels of maximum LV volume, minimum LV volume, end-diastolic pressure, and a measurement of systolic pressure. The present inventor found the error landscape for this optimization to be very well-suited to automatic parameter identification (FIG. 7, Table 3). Using the built-in fminsearch function in MATLAB, our automatic tuning algorithm required one hour of computation time, but it should be fairly straightforward to reduce this time substantially.

Interestingly, most published growth modeling studies have paid less attention to quantitatively matching hemodyanmic loading than the present study, but have employed much more realistic representations of left ventricular geometry. Realistic geometries are now relatively easy to obtain in a clinical setting using MRI or CT. Yet, translating these images into a finite-element mesh typically requires some user intervention in the segmentation and assembly pipeline, adding to the time required to customize the model for a specific patient. One implication of our results is that for some conditions such as global pressure or volume overload, detailed representations of left ventricular geometry may not be essential for predicting the time course of growth. Additionally, since geometrically simpler models require only a few measurements (LV diameter and thickness) that are easily obtained from echocardiography, using such models could reduce imaging time and cost.

On the other hand, when modeling cardiac growth and remodeling, it is essential to account for how geometric changes feedback to influence LV function. For example, the same active myofiber stress translates to much lower pressures in a dilated, thin-walled heart compared to a heart with a normal geometry. One advantage of finite-element models is that they account for these geometric effects automatically, since they specify force generation at the myofiber or single-element level and integrate over the mesh to determine the corresponding cavity pressure. Here, in an embodiment, the present inventor used a time-varying elastance model of ventricular contraction, which required us to modify the governing diastolic and systolic pressure-volume relationships to account for changes in geometry as remodeling progressed. In an embodiment, the present inventor did this by developing analytic expressions based on the relationships between strain and volume and stress and pressure in a thin-walled sphere. Clearly, the heart is not a thin-walled sphere, and stresses estimated using formulas for a thin-walled sphere are not very accurate. Yet, this limitation did not impair our ability to predict growth-related changes in passive or active chamber properties, likely because relative changes in radius and wall thickness have similar relative impacts on stress in simpler and more complex cardiac geometries.

Finally, the growth law employed here was originally implemented in a finite element model and prescribed the same amount of growth in the radial and cross-fiber directions. The spherical model only has two directions (circumferential and radial), which means it cannot simulate changes in LV shape that might arise from differential growth in the fiber and cross-fiber directions. This limitation did not seem to diminish the ability of the model to predict ventricular dilation or thickening in response to global hemodyanmic overload,

The system, method and computer readable medium described herein includes, among other things, multiple components that are necessary to predict growth and remodeling of the heart from patient data.

The computational model described and provided herein includes, among other things, multiple components that are necessary to predict growth and remodeling of the heart from patient data, such as but not limited thereto, the following:

1) An algorithm (and system, method and computer readable medium) that correctly predicts future growth and remodeling of the heart (defined as changes in mass, dimensions, shape, volume, wall thickness, and other geometric features) in response to many different diseases including but not limited to hypertension, aortic stenosis, mitral regurgitation, myocardial infarction, left bundle branch block, and other conditions that alter the mechanics of the heart.

2) A method (and system and computer readable medium) for calibrating the growth algorithm against a historical dataset in order to predict growth and remodeling for any specific disease or condition.

3) A computational implementation (and system, method and computer readable medium) that simulates many months of heart growth and remodeling in just a few seconds on a laptop computer. This fast computational speed is an enabling advance that is essential for making predictions during a procedure or for simulating many possible options as part of treatment planning for individual patients (see potential uses below).

4) A method (and system and computer readable medium) for automatically fitting the computational model to data from an individual patient in order to make patient-specific predictions.

5) A method (and system and computer readable medium) for using that automated parameter fitting process to noninvasively estimate key variables that are difficult or impossible to measure directly in patients, including but not limited to the contractility of undamaged myocardium following myocardial infarction and the degree of venoconstriction.

These variables have potential prognostic value in many different settings beyond their value in predicting future growth and remodeling of the heart.

An aspect of an embodiment provides, among other things, a system, method and computer readable medium for predicting the time course of ventricular dilation and thickening using a rapid compartmental model.

An aspect of an embodiment includes a system, method and computer readable medium for providing, among other things, rapid models of cardiac growth and remodeling.

An aspect of an embodiment of the present invention provides, but not limited thereto, a computer-implemented method for rapidly predicting cardiac response to a heart condition and treatment strategy of a subject using a compartmental model. The method may comprise: receiving disease-specific data; calibrating the compartmental model based on the disease-specific data; receiving patient-specific data; tuning parameters using the patient-specific data; simulating the treatment strategy using the tuned parameters with patient-specific data; and predicting cardiac response, for use on the subject, using the simulated treatment strategy and the disease-specific-calibrated model.

An aspect of an embodiment of the present invention provides, but not limited thereto, a method for determining cardiovascular information of subject. The method may comprise: receiving patient-specific data; tuning parameters using the patient-specific data; and generating patient-specific prognostic data from the tuned parameters for use on the subject.

An aspect of an embodiment of the present invention provides, but not limited thereto, a system for rapidly predicting cardiac response to a heart condition and treatment strategy of a subject using a compartmental model. The system may comprise a memory for storing instructions and a processor configured to execute the instructions to: receive disease-specific data; calibrate the compartmental model based on the disease-specific; receive patient-specific data; tune parameters using the patient-specific data; simulate the treatment strategy using the tuned parameters with patient-specific data; and predict cardiac response, for use on the subject, using the simulated treatment strategy and the disease-specific-calibrated model.

An aspect of an embodiment of the present invention provides, but not limited thereto, a system for determining cardiovascular information of subject. The system may comprise a memory for storing instructions and a processor configured to execute the instructions to: receive patient-specific data; tune parameters using the patient-specific data; and generate patient-specific prognostic data from the tuned parameters for use on the subject.

An aspect of an embodiment of the present invention provides, but not limited thereto, a computer program product comprising a non-transitory computer readable storage medium containing computer-executable instructions for rapidly predicting cardiac response to a heart condition and treatment strategy of a subject using a compartmental model. The instructions causing a computer to: receive disease-specific data; calibrate the compartmental model based on the disease-specific; receive patient-specific data; tune parameters using the patient-specific data; simulate the treatment strategy using the tuned parameters with patient-specific data; and predict cardiac response, for use on the subject, using the simulated treatment strategy and the disease-specific-calibrated model.

An aspect of an embodiment of the present invention provides, but not limited thereto, a computer program product comprising a non-transitory computer readable storage medium containing computer-executable instructions for determining cardiovascular information of subject. The instructions causing a computer to: receive patient-specific data; tune parameters using the patient-specific data; and generate patient-specific prognostic data from the tuned parameters for use on the subject.

It should be appreciated that any of the components or modules referred to with regards to any of the present invention embodiments discussed herein, may be integrally or separately formed with one another. Further, redundant functions or structures of the components or modules may be implemented. Moreover, the various components may be communicated locally and/or remotely with any user/clinician/patient or machine/system/computer/processor. Moreover, the various components may be in communication via wireless and/or hardwire or other desirable and available communication means, systems and hardware. Moreover, various components and modules may be substituted with other modules or components that provide similar functions.

It should be appreciated that the device and related components discussed herein may take on all shapes along the entire continual geometric spectrum of manipulation of x, y and z planes to provide and meet the anatomical, environmental, and structural demands and operational requirements. Moreover, locations and alignments of the various components may vary as desired or required.

It should be appreciated that various sizes, dimensions, contours, rigidity, shapes, flexibility and materials of any of the components or portions of components in the various embodiments discussed throughout may be varied and utilized as desired or required.

It should be appreciated that while some dimensions are provided on the aforementioned figures, the device may constitute various sizes, dimensions, contours, rigidity, shapes, flexibility and materials as it pertains to the components or portions of components of the device, and therefore may be varied and utilized as desired or required.

Although example embodiments of the present disclosure are explained in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g. rat, dog, pig, monkey), etc. It should be appreciated that the subject may be any applicable human patient, for example.

As discussed herein, a “subject” may be any applicable human, animal, or other organism, living or dead, or other biological or molecular structure or chemical environment, and may relate to particular components of the subject, for instance specific tissues or fluids of a subject (e.g., human tissue in a particular area of the body of a living subject), which may be in a particular location of the subject, referred to herein as an “area of interest” or a “region of interest.”

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the n^{th} reference in the list. All references cited and discussed in this specification are incorporated herein by reference in their entireties and to the same extent as if each reference was individually incorporated by reference.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

A method and system for rapidly predicting cardiac response to a heart condition and treatment strategy of a patient. Predictions are accomplished using a compartmental model that includes systemic and pulmonary circulations represented as a system of resistors and capacitors together with chambers of the heart represented as simple geometric shapes such as spheres or assemblies of spheres or other analytic equations that relate pressure and volume to stress and strain. The model is calibrated using disease-specific data sets for a specific heart condition. Simple geometry modeling allows for rapid use such that hemodynamic parameters may be tuned for optimal accuracy. Patient-specific data is received through an acquisition device, such as imaging or diagnostic devices and hemodynamic parameters are tuned based on patient-specific data such that tuned parameters are optimized to a specific patient. Patient-specific tuned parameters are used as inputs to simulate in real-time (or other specified duration) a patient-specific treatment strategy. Cardiac response is then predicted based on the patient-specific treatment strategy using the calibrated compartmental model. Predicted cardiac response is evaluated for optimal outcomes. Treatment strategies may be modified and re-simulated in real time if necessary to achieve a more optimal outcome and treatment. Cardiac response may be predicted based on the modified and re-simulated treatment strategy. Additional iterations of evaluating, modifying, re-simulating, and predicting in real time (or other specified durations) may be used to achieve a more optimal or acceptable outcome. The treatment strategy related to the acceptable predicted cardiac response is outputted to inform the patient-specific treatment strategy. Tuning hemodynamic parameters also generates patient-specific prognostic data that is otherwise impossible to measure and provides important patient-specific cardiovascular information for further use on patient.

For example, FIGS. 1-4 and 13 illustrate a method and/or system for rapidly predicting cardiac response to a heart condition and treatment strategy of a subject whereby the related system, processor, modules, hardware, and firmware is operated in in real time using a compartmental model according to an embodiment of the present invention.

For example, FIGS. 1-4 and 13 illustrate a method and/or system for rapidly predicting cardiac response to a heart condition and treatment strategy of a subject for a specified duration whereby the related system, processor, modules, hardware, and firmware is operated in a range greater than zero seconds and less than one second using a compartmental model according to an embodiment of the present invention.

For example, FIGS. 1-4 and 13 illustrate a method and/or system for rapidly predicting cardiac response to a heart condition and treatment strategy of a subject for a specified duration whereby the related system, processor, modules, hardware, and firmware is operated in a range greater than zero seconds and less than two seconds using a compartmental model according to an embodiment of the present invention.

For example, FIGS. 1-4 and 13 illustrate a method and/or system for rapidly predicting cardiac response to a heart condition and treatment strategy of a subject for a specified duration whereby the related system, processor, modules, hardware, and firmware is operated in a range greater than zero seconds and less than five seconds using a compartmental model according to an embodiment of the present invention.

For example, FIGS. 1-4 and 13 illustrate a method and/or system for rapidly predicting cardiac response to a heart condition and treatment strategy of a subject for a specified duration whereby the related system, processor, modules, hardware, and firmware is operated in a range greater than zero seconds and less than ten seconds using a compartmental model according to an embodiment of the present invention.

For example, FIGS. 1-4 and 13 illustrate a method and/or system for rapidly predicting cardiac response to a heart condition and treatment strategy of a subject for a specified duration whereby the related system, processor, modules, hardware, and firmware is operated in a range greater than zero seconds and less than thirty seconds using a compartmental model according to an embodiment of the present invention.

For example, FIGS. 1-4 and 13 illustrate a method and/or system for rapidly predicting cardiac response to a heart condition and treatment strategy of a subject for a specified duration whereby the related system, processor, modules, hardware, and firmware is operated in a range greater than zero seconds and less than one minute using a compartmental model according to an embodiment of the present invention.

For example, FIGS. 1-4 and 13 illustrate a method and/or system for rapidly predicting cardiac response to a heart condition and treatment strategy of a subject for a specified duration whereby the related system, processor, modules, hardware, and firmware is operated in a range greater than zero seconds and less than two minutes using a compartmental model according to an embodiment of the present invention.

For example, FIGS. 1-4 and 13 illustrate a method and/or system for rapidly predicting cardiac response to a heart condition and treatment strategy of a subject within a specified time range using a compartmental model according to an embodiment of the present invention, wherein said time range includes the related system, processor, modules, hardware, and firmware operating in a specified duration of anyone of the following ranges: greater than zero seconds and less than about 1 second; greater than zero seconds and less than about two seconds; greater than zero seconds and less than about five seconds; greater than zero seconds and less than about ten seconds; greater than zero seconds and less than about thirty seconds; greater than zero seconds and less than about 1 minute; greater than zero seconds and less than about 2 minutes; greater than zero seconds and less than about 5 minutes; greater than zero seconds and less than about 15 minutes; greater than zero seconds and less than about 30 minutes; greater than zero seconds and less than about an hour; greater than zero seconds and less than about two hours; greater than zero seconds and less than about four hours; greater than zero seconds and less than about twelve hours; greater than zero seconds and less than about twenty-four hours; or greater than zero seconds and less than one week. The numerical ranges recited herein by endpoints include all numbers and fractions subsumed within that range. Similarly, numerical ranges recited herein by endpoints include subranges subsumed within that range. It should be appreciated that the specified duration may be greater than twenty four hours. It should be appreciated that the specified duration may be greater one week.

The invention itself, together with further objects and attendant advantages, will best be understood by reference to the following detailed description, taken in conjunction with the accompanying drawings.

These and other objects, along with advantages and features of various aspects of embodiments of the invention disclosed herein, will be made more apparent from the description, drawings and claims that follow.

### BRIEF DESCRIPTION OF THE DRAWINGS

The foregoing and other objects, features and advantages of the present invention, as well as the invention itself, will be more fully understood from the following description of preferred embodiments, when read together with the accompanying drawings.

The accompanying drawings, which are incorporated into and form a part of the instant specification, illustrate several aspects and embodiments of the present invention and, together with the description herein, serve to explain the principles of the invention. The drawings are provided only for the purpose of illustrating select embodiments of the invention and are not to be construed as limiting the invention.

FIG. 1 illustrates a method and/or system for rapidly predicting cardiac response to a heart condition and treatment strategy of a subject using a compartmental model according to an embodiment of the present invention.

FIG. 2 illustrates a method and/or system for rapidly predicting cardiac response to a heart condition and treatment strategy of a subject using a compartmental model wherein said predicted cardiac response and said treatment strategy are evaluated and modified according to an embodiment of the present invention.

FIG. 3 illustrates a method and/or system for rapidly predicting cardiac response to a heart condition and treatment strategy of a subject using a compartmental model wherein said predicted cardiac response and said treatment strategy are evaluated and modified, and patient-specific prognostic data is generated from tuning hemodynamic parameters or other parameters using patient-specific data according to an embodiment of the present invention.

FIG. 4 illustrates a method and/or system for rapidly determining cardiovascular information of a subject by generating patient-specific prognostic data from tuning hemodynamic parameters or other parameters using patient-specific data according to an embodiment of the present invention.

FIG. 5 is a block diagram illustrating an example of a machine upon which one or more aspects of embodiments of the present invention can be implemented.

FIG. 6 illustrates a schematic representation modeling systemic and pulmonary circulations that are represented as a system of resistors and capacitors used to simulate the pressure-volume behavior of the cardiovascular system.

FIG. 7 visually represents the sensitivity analysis of tuned hemodynamic parameter sets against previously published experimental data [See Kleaveland, J. P., Kussmaul, W. G., Vinciguerra, T., Diters, R., & Carabello, B. A. (1988). Volume overload hypertrophy in a closedchest model of mitral regurgitation. The American Journal of Physiology, 254(6 Pt 2), H1034-H1041] and literature regarding the physiologic values of end-systolic stretch relative to an unloaded state [See Witzenburg, C. M., & Holmes, J. W. (2017). A comparison of phenomenologic growth laws for myocardial hypertrophy. Journal of Elasticity, 129(1-2), 257-281]. FIG. 7A visually represents mean squared error (MSE) in Z scores computed as the unloaded volume of the ventricle (V₀) and systemic vascular resistance (SVR_{control}) were varied between 50 and 150% of their optimized values with all other parameters set to their optimized values and where changes in either parameter greater than 15% pushed MSE above 0.04. FIG. 7B visually represents MSE computed as V₀ and end-systolic elastance of the ventricle (E) were varied in the same manner and where many combinations of V₀ and E produced MSE values below 0.04. FIG. 7C-D visually represents that repeating the simulations from FIGS. 7A-B using an augmented objective function provided a unique optimum for all parameters including V₀ and E.

FIG. 8 graphically represents the pressure-volume relationship of the LV remained similar to baseline and acutely thereafter the onset of LBBB, but shifted to the right following growth to chronic state.

FIG. 9A graphically represents the predicted increases (as represented by the line generally traveling left-to-right in the graphical illustration) in LV EDV versus time. FIG. 9B graphically represents the wall volume, as represent by wall volume change in percentage, (as represented by the line generally traveling left-to-right in the graphical illustration fell well within the range of the standard deviation of previously published experimental data

[See Vernooy et al., Eur Heart J, 26(1): 91-98, 2005]

FIGS. 10A-10B graphically represents simulated activation maps of LV segments for non-ischemic for LBBB and CRT, respectively. The star (asterisk) of FIG. 10B indicates LV lead location, and the crosses (X's) of FIG. 10A the position of simulated lateral-midwall ischemia.

FIGS. 11A-11B graphically represents the calibrated model matched changes in (a) lateral and septal wall mass (See FIG. 11A) and EDV of experimental results (See FIG. 11B) [See Vernooy et al., Eur. Heart J., 28(17): 2148-2155, 2007].

FIGS. 12A-12B graphically represents the pacing locations influenced CRT outcomes for non-ischemia and ischemic LBBB, respectively.

FIG. 13 illustrates a method and/or system for rapidly predicting cardiac response to a heart condition and treatment strategy of a subject using a compartmental model wherein said systemic and pulmonary circulations are represented as a system of resistors and capacitors and said chambers of the heart are represented as spheres or assemblies of spheres or other analytic equations that relate pressure and volume to stress and strain.

FIG. 14 is a high-level block diagram of a computer (or other machine) capable of implementing an aspect of embodiment of the present invention.

### DETAILED DESCRIPTION OF EXEMPLARY EMBODIMENTS

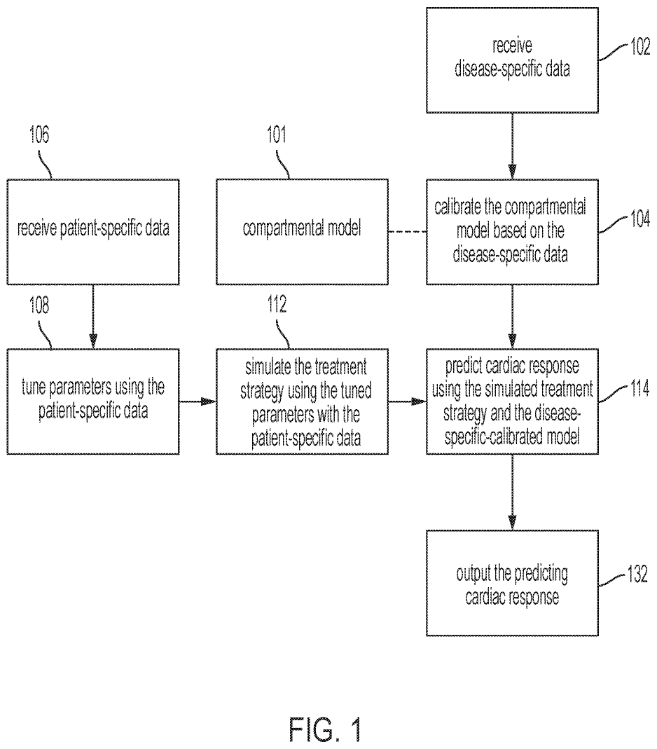

The elements and features (i.e., components) illustrated in FIGS. 1-4 and 13, and similarly throughout this disclosure, are representative of method steps, computer-implemented method steps, hardware modules and/or software modules. Although only a limited number of each of the elements and features (i.e., components) are depicted, this is for illustrative purposes only and is not to be construed as limiting. It is to be understood that the components depicted may be logical components and that the terminology used herein to describe each component is for illustrative purposes and is not to be construed as limiting. Components are herein referenced as “systems,” “mechanisms,” “methods”, “modules,” “processors,” etc. Each component may include the necessary method steps, apparatuses, hardware, firmware, and/or software to enable the processing, storing, communicating and/or receiving of data. A component may include one or more computer processors, computer servers, data stores, electronic components, storage mediums, memory, etc. The functionality of a component may be directed by one or more executable computer-readable instructions received via a computer-readable storage medium. A processor may be included to execute one or more functions per instructions, programs, or processes stored in the processor itself and/or stored in another memory source. Memory may be any mechanism that is capable of storing data, such as computer programs, instructions, and other necessary data. One or more interfaces may be included to enable the presentation, manipulation, transmission, and receipt of data. Communication of data may be enabled by one or more networks or physical connections. A network may include one or more of a wide-area network (WAN) (such as the Internet), a local area network (LAN), a wireless local area network (WLAN), a mobile wireless network a combination of any of the foregoing, or any other suitable network and may include any component (physical or logical) necessary for a particular network's functionality, such as routers, adapters, subnets, etc.

In an embodiment, at any one or more of the disclosed steps, hardware modules and/or software modules such steps or modules may be in communication with one or more output devices, storage devices, displays, computers, networks, machines, or other systems or device as desired or required as well as in communication with users. Thus, the steps of the methods or modules of the systems (or modules of software) of FIGS. 1-4 and 13 may be defined by the computer program instructions stored in the memory 1410 and/or storage 1412 and controlled by the processor 1404 executing the computer program instructions.

FIG. 1 illustrates a method and/or system for rapidly predicting cardiac response to a heart condition and treatment strategy of a subject using a compartmental model according to an embodiment of the present invention. Again, elements and features (i.e., components) illustrated in FIGS. 1, and similarly throughout this disclosure, are representative of method steps, computer-implemented method steps, hardware modules and/or software modules.

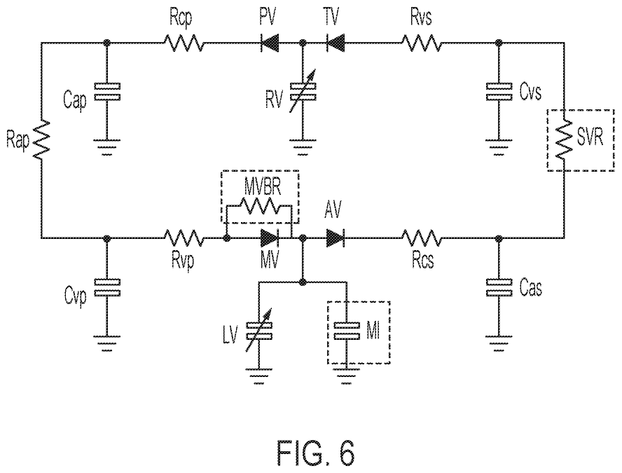

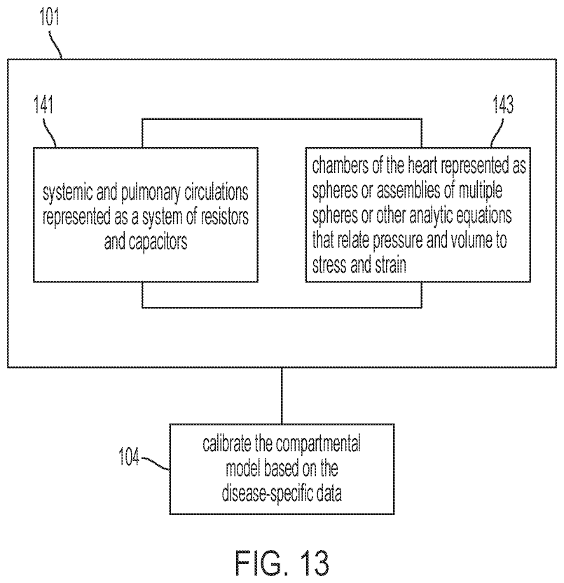

Referring to FIG. 1, a compartmental model 101 may be comprised of systemic and pulmonary circulations represented as a system of resistors and capacitors 141. For example, in FIG. 6 systemic and pulmonary circulations are represented as a system of resistors and capacitors and can be used to simulate the pressure-volume behavior of the cardiovascular system. Compartmental model 101 may include chambers of the heart represented as spheres or assemblies of spheres (or substantially spherical or assemblies of substantially spherical shapes) or other analytic equations that relate pressure and volume to stress and strain 143 as seen in FIG. 13.

At step 102, the system then receives disease-specific data from step, for example from a historical data set. The compartmental model 101 is then calibrated at step 104 based on the disease specific data received in step 102.

At step 106, the system receives patient-specific data that will be used to simulate growth and treatment. For example, patient-specific data may be received through an acquisition device 1420. An acquisition device may be an imaging device that includes at least one or more of the following: magnetic resonance imaging (MRI), ultrasound, computed tomography (CT), positron emission tomography (PET), electroanatomic mapping device, or nuclear imaging. An acquisition device may also include a diagnostic device including at least one of the following: electrocardiogram (ECG or EKG), other cardiac electrical data device, or diagnostic devices. Patient-specific data may include at least one or any combination of the following: hemodynamic data; anatomic or functional imaging data from

MRI, ultrasound, CT, PET, nuclear or other imaging modalities; ECG, inverse ECG, electroanatomic mapping, or any other cardiac electrical data; medical history; current and past medications; or any other patient-specific information that could affect predictions of heart responses.

At step 108, an aspect an embodiment of the present invention tunes parameters related to hemodynamic data using patient-specific data acquired in step 106 to find optimized patient-specific tuned parameters. The tuned parameters may include at least one or any combination of the following: hemodynamic data or other specified data as desired or required

At step 112, an aspect of an embodiment of the present invention then simulates the treatment strategy using the tuned parameters based on patient-specific data 108.

At step 114, an aspect an embodiment of the present invention then predicts a given cardiac response using the disease-specific-calibrated model 104 and simulated treatment strategy 112 based on the parameters tuned using patient-specific data 108. A cardiac response may include at least one or any combination of the following: changes in heart dimensions, mass, or cavity volumes including growth, hypertrophy, remodeling, shrinkage, or atrophy; changes in heart composition including fibrosis; and changes in heart function including improved or diminished ejection fraction, stroke work, contractility, valvular regurgitation, and synchrony or dyssnchrony of contraction.

An aspect of an embodiment of the present invention may then output the predicted cardiac response 132.

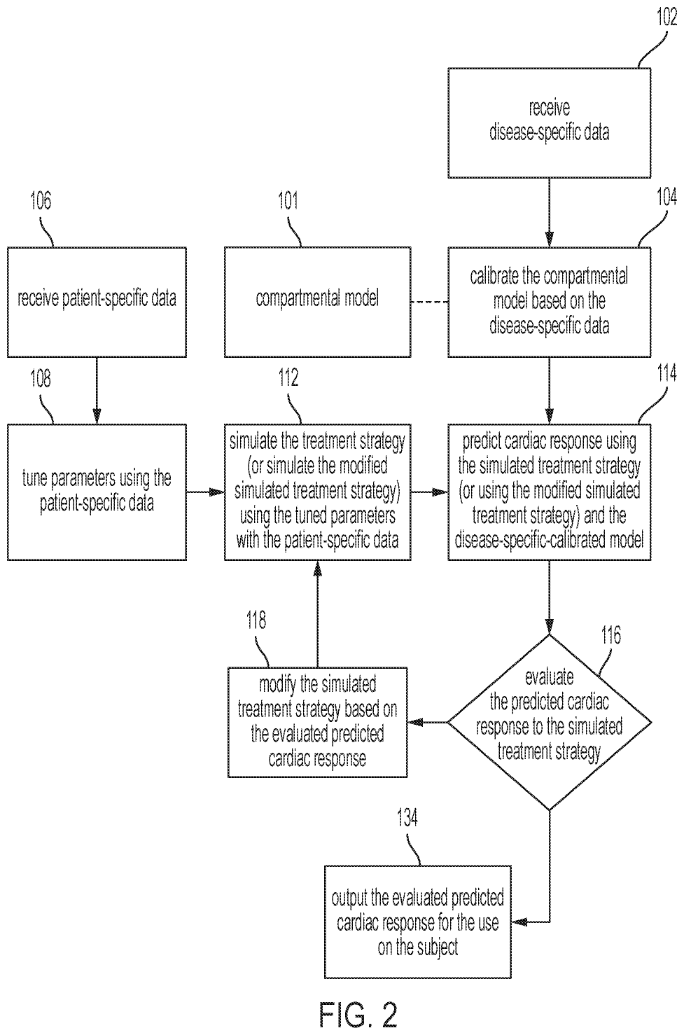

FIG. 2 illustrates a method and/or system for rapidly predicting cardiac response to a heart condition and treatment strategy of a subject using a compartmental model wherein said predicted cardiac response and said treatment strategy are evaluated and modified according to an embodiment of the present invention. Again, elements and features (i.e., components) illustrated in FIGS. 2, and similarly throughout this disclosure (e.g. FIGS. 1-4 and 13, etc.), are representative of method steps, computer-implemented method steps, hardware modules and/or software modules.

Still referring to FIG. 2, in accordance with the method and/or system of an embodiment of the present invention illustrated in FIG. 1, at step 116, an aspect of an embodiment of the present invention may evaluate the predicted cardiac response of 114 to determine whether the predicted cardiac response is an optimal or acceptable outcome. If upon evaluation (at step 116) the predicted cardiac response 114 is not an optimal or acceptable response, an aspect of an embodiment of the present invention may modify the simulated treatment strategy 118 based on the evaluated predicted cardiac response. The modified simulated treatment strategy 118 may then be re-simulated as in step 112 (e.g., simulating, in step 112, the modified simulated treatment strategy from step 118) using the parameters tuned with the patient-specific data from step 108. An aspect of an embodiment of the present invention may use the re-simulated treatment strategy to predict a more optimal or acceptable cardiac response as in step 114 (e.g., predicting cardiac response, for use on the subject, using the modified simulated treatment strategy). Additional iterations of evaluating, modifying, re-simulating, and predicting may be used to achieve a more optimal or acceptable outcome.

At step 134, if the evaluation of the predicted cardiac response is an optimal or acceptable outcome (from step 116), then the evaluated predicted cardiac response 116 may be outputted for use on a subject, at step 134. In an aspect of an embodiment of the present invention, the predicted cardiac response of 114 may then cause a user, which may include a technician, clinician, or physician, to take action on a subject based on the simulated treatment strategy and predicted cardiac response in relation to said simulated treatment strategy.

In an embodiment, referring to step or module 116, such step may be implemented manually by a user (rather than by the processor, for example) for evaluating the predicted cardiac response of 114 to determine whether the predicted cardiac response is an optimal or acceptable outcome, etc. Similarly, in an embodiment, referring to step or module 118, such step may be implemented manually by a user (rather than by the processor, for example) for modifying the simulated treatment strategy 118 based on the evaluated predicted cardiac response of step or module 116.

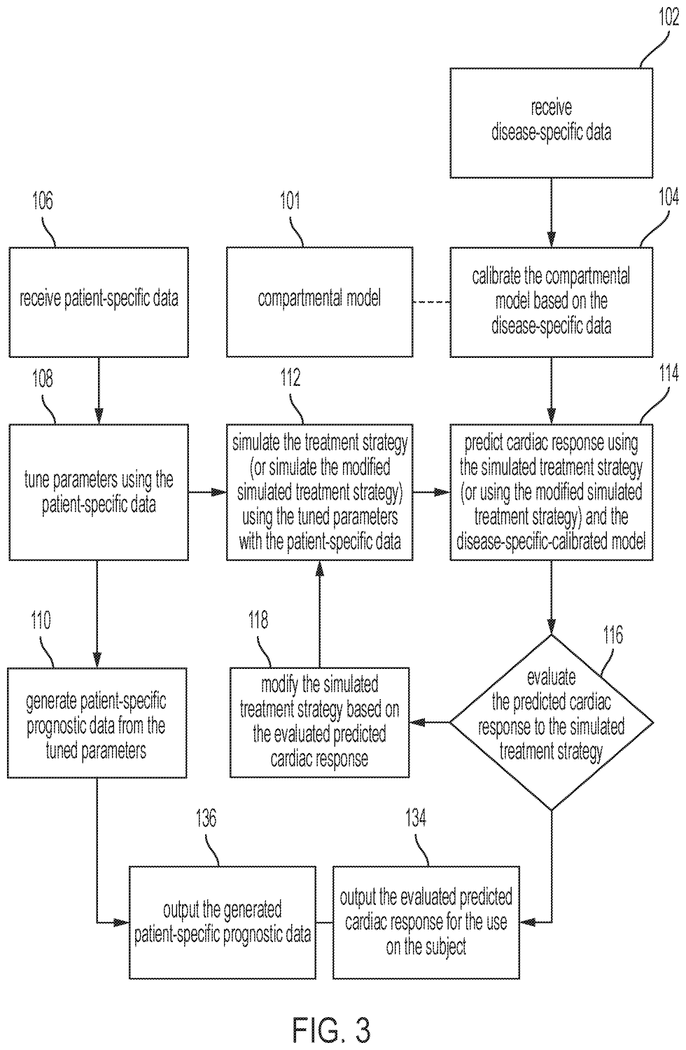

FIG. 3 illustrates a method and/or system for rapidly predicting cardiac response to a heart condition and treatment strategy of a subject using a compartmental model wherein said predicted cardiac response and said treatment strategy are evaluated and modified, and patient-specific prognostic data is generated from tuning hemodynamic parameters or other parameters using patient-specific data according to an embodiment of the present invention.

Still referring to FIG. 3, in accordance with the method and/or system of an embodiment of the present invention illustrated in FIG. 1-2, at step 110, an aspect an embodiment of the present invention may generate patient-specific prognostic data from the parameters tuned using patient-specific data in 108. The parameters tuned using patient specific-data 108 are optimized in such a way that generates unique patient-specific prognostic-data. The generated patient-specific prognostic data 110 includes some data impossible to measure, such as measures of contractility of undamaged myocardium following myocardial infarction and the contractility of individual subregions of the heart in the presence of dyssynchrony. The generated patient-specific prognostic data 110 may also include noninvasive measures of the contractility of myocardium in any disease or condition. The generated patient-specific prognostic data 110 may also include measures of the degree of venoconstriction and measures of the totally blood volume and fluid status.

At step 136, the patient-specific prognostic data, including some data impossible to other wise measure, may be outputted for use on a subject. In an aspect an embodiment of the present invention, the patient-specific prognostic data of 110 may then cause a user, which may include a technician, clinician, or physician, to take action on a subject based on this prognostic data.

Still referring to FIG. 3, an embodiment may be implemented without: steps 116, 118 or 134; steps 118 or 134; or step 118.

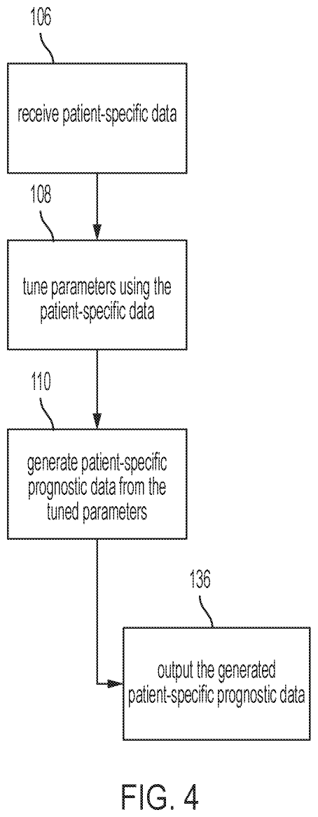

FIG. 4 illustrates a method and/or system for rapidly determining cardiovascular information of a subject by generating patient-specific prognostic data from tuning hemodynamic parameters or other parameters using patient-specific data according to an embodiment of the present invention.

At step 106, the system receives patient-specific data. For example, patient-specific data may be received through an acquisition device 1420. An acquisition device may be an imaging device that includes at least one or more of the following: magnetic resonance imaging (MRI), ultrasound, computed tomography (CT), positron emission tomography (PET), electroanatomic mapping device, or nuclear imaging. An acquisition device may also include a diagnostic device including at least one of the following: electrocardiogram (ECG or EKG), other cardiac electrical data device, or diagnostic devices. Patient-specific data may include at least one or any combination of the following: hemodynamic data; anatomic or functional imaging data from MRI, ultrasound, CT, PET, nuclear or other imaging modalities; ECG, inverse ECG, electroanatomic mapping, or any other cardiac electrical data; medical history; current and past medications; or any other patient-specific information that could affect predictions of heart responses.

At step 108, an aspect an embodiment of the present invention tunes parameters related to hemodynamic data using patient-specific data acquired in step 106 to find optimized patient-specific tuned parameters. The tuned parameters may include at least one or any combination of the following: hemodynamic data or other specified data as desired or required

At step 110, an aspect of an embodiment of the present invention may generate patient-specific prognostic data from the parameters tuned using patient-specific data in 108. The parameters tuned using patient specific-data 108 are optimized in such a way that generates unique patient-specific prognostic-data. The generated patient-specific prognostic data 110 includes some data impossible to measure, such as measures of contractility of undamaged myocardium following myocardial infarction and the contractility of individual subregions of the heart in the presence of dyssynchrony. The generated patient-specific prognostic data 110 may also include noninvasive measures of the contractility of myocardium in any disease or condition. The generated patient-specific prognostic data 110 may also include measures of the degree of venoconstriction and measures of the totally blood volume and fluid status.

At step 136, the patient-specific prognostic data, including some data impossible to otherwise measure, may be outputted for use on a subject. In an aspect of an embodiment of the present invention, the patient-specific prognostic data of 110 may then cause a user, which may include a technician, clinician, or physician, to take action on a subject based on this prognostic data.

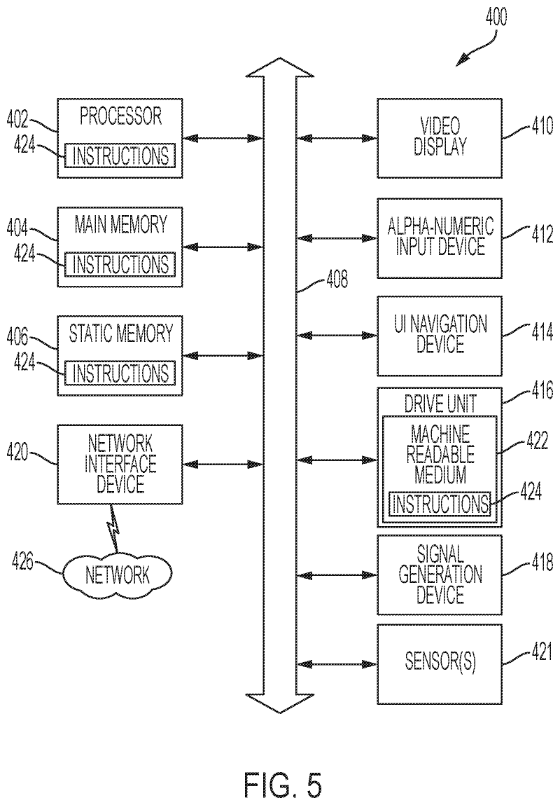

FIG. 5 is a block diagram illustrating an example of a machine upon which one or more aspects of embodiments of the present invention can be implemented.

FIG. 5 represents an aspect of an embodiment of the present invention relating to a system, method and computer readable medium for: a) predicting the time course of ventricular dilation and thickening, b) modeling cardiac responses to heart conditions and simulated treatments, and/or c) rapidly modeling a disease-specific cardiac response to a heart condition and predicting cardiac response to a patient-specific treatment strategy based on tuned hemodynamic parameters or other parameters, which illustrates a block diagram of an example machine 400 upon which one or more embodiments (e.g., discussed methodologies) can be implemented (e.g., run).

Examples of machine 400 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory machine readable medium or (2) in a transmission signal. In an example, the software, when executed by the underlying hardware of the circuit, causes the circuit to perform the certain operations.

In an example, a circuit can be implemented mechanically or electronically. For example, a circuit can comprise dedicated circuitry or logic that is specifically configured to perform one or more techniques such as discussed above, such as including a special-purpose processor, a field programmable gate array (FPGA) or an application-specific integrated circuit (ASIC). In an example, a circuit can comprise programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In embodiments in which multiple circuits are configured or instantiated at different times, communications between such circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor-implemented. For example, at least some of the operations of a method can be performed by one or processors or processor-implemented circuits. The performance of certain of the operations can be distributed among the one or more processors, not only residing within a single machine, but deployed across a number of machines. In an example, the processor or processors can be located in a single location (e.g., within a home environment, an office environment or as a server farm), while in other examples the processors can be distributed across a number of locations.

The one or more processors can also operate to support performance of the relevant operations in a “cloud computing” environment or as a “software as a service” (SaaS). For example, at least some of the operations can be performed by a group of computers (as examples of machines including processors), with these operations being accessible via a network (e.g., the Internet) and via one or more appropriate interfaces (e.g., Application Program Interfaces (APIs).)

Example embodiments (e.g., apparatus, systems, or methods) can be implemented in digital electronic circuitry, in computer hardware, in firmware, in software, or in any combination thereof. Example embodiments can be implemented using a computer program product (e.g., a computer program, tangibly embodied in an information carrier or in a machine readable medium, for execution by, or to control the operation of, data processing apparatus such as a programmable processor, a computer, or multiple computers).

A computer program can be written in any form of programming language, including compiled or interpreted languages, and it can be deployed in any form, including as a stand-alone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., a field programmable gate array (FPGA) or an application-specific integrated circuit (ASIC)).

The computing system can include clients and servers. A client and server are generally remote from each other and generally interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine 400) and software architectures that can be deployed in example embodiments.

In an example, the machine 400 can operate as a standalone device or the machine 400 can be connected (e.g., networked) to other machines.

In a networked deployment, the machine 400 can operate in the capacity of either a server or a client machine in server-client network environments. In an example, machine 400 can act as a peer machine in peer-to-peer (or other distributed) network environments. The machine 400 can be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 400. Further, while only a single machine 400 is illustrated, the term “machine” shall also be taken to include any collection of machines that individually or jointly execute a set (or multiple sets) of instructions to perform any one or more of the methodologies discussed herein.

Example machine (e.g., computer system) 400 can include a processor 402 (e.g., a central processing unit (CPU), a graphics processing unit (GPU) or both), a main memory 404 and a static memory 406, some or all of which can communicate with each other via a bus 408. The machine 400 can further include a display unit 410, an alphanumeric input device 412 (e.g., a keyboard), and a user interface (UI) navigation device 411 (e.g., a mouse). In an example, the display unit 410, input device 417 and UI navigation device 414 can be a touch screen display. The machine 400 can additionally include a storage device (e.g., drive unit) 416, a signal generation device 418 (e.g., a speaker), a network interface device 420, and one or more sensors 421, such as a global positioning system (GPS) sensor, compass, accelerometer, or other sensor.

The storage device 416 can include a machine readable medium 422 on which is stored one or more sets of data structures or instructions 424 (e.g., software) embodying or utilized by any one or more of the methodologies or functions described herein. The instructions 424 can also reside, completely or at least partially, within the main memory 404, within static memory 406, or within the processor 402 during execution thereof by the machine 400. In an example, one or any combination of the processor 402, the main memory 404, the static memory 406, or the storage device 416 can constitute machine readable media.

While the machine readable medium 422 is illustrated as a single medium, the term “machine readable medium” can include a single medium or multiple media (e.g., a centralized or distributed database, and/or associated caches and servers) that configured to store the one or more instructions 424. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media. Specific examples of machine readable media can include non-volatile memory, including, by way of example, semiconductor memory devices (e.g., Electrically Programmable Read-Only Memory (EPROM), Electrically Erasable Programmable Read-Only Memory (EEPROM)) and flash memory devices;

magnetic disks such as internal hard disks and removable disks; magneto-optical disks; and CD-ROM and DVD-ROM disks.

The instructions 424 can further be transmitted or received over a communications network 426 using a transmission medium via the network interface device 420 utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

FIG. 6 illustrates a method and/or system for modeling systemic and pulmonary circulations that are represented as a system of resistors and capacitors used to simulate the pressure-volume behavior of the cardiovascular system. In an embodiment, this system of resistors and capacitors can be considered a portion of a compartmental model 101 (as disclosed herein or disclosed in FIG. 13).

FIG. 6 illustrates a schematic of the circuit model used to simulate the pressure-volume behavior of the cardiovascular system. Systemic and pulmonic circuits consisted of a characteristic resistance (R_{cs} and R_{cp}), arterial resistance (SVR and Rₐₚ), resistance to venous return (Rᵥₛ and Rᵥₚ), arterial compliance (Cₐₛ and Cₐₚ), and venous compliance (Cᵥₛ and Cᵥₚ), respectively. Pressure sensitive diodes (TV, PV, MV, and AV) respresented the tricuspid, pulmonary, mitral, and aortic valves, respectively. Elements outlined in dashed lines were added or altered in order to simulate hemodynamic overload. An increase in SVR simulated aortic occlusion, a decrease in the backflow resistance of the mitral valve (MVBR) simulated mitral valve regurgitation, and dividing the left ventricle (LV) into active and passive compartments (MI) simulated myocardial infarction.

A model of the ventricles and circulation similar to that employed by Santamore and Burkhoff [See Santamore, W. P., & Burkhoff, D. (1991). Hemodynamic consequences of ventricular interaction as assessed by model analysis. The American Journal of Physiology, 260(1 Pt 2), H146-H157] was used to simulate hemodynamics throughout the cardiac cycle. In this model (FIG. 6) the ventricles were simulated using time-varying elastances. The left ventricular end-diastolic and end-systolic pressure-volume relationships were defined by

P_{ES}=E*(V_{ES}−V₀) (1)

P_{ED}B*exp[A*(V_{ED}−V₀)]B (2)

respectively, where E was the end-systolic elastance of the ventricle, V₀ was the unloaded volume of the ventricle, and A and B were coefficients describing the exponential shape of the end-diastolic pressure-volume relationship (EDPVR). The present inventor assumed the same end-diastolic parameters for the right and left ventricles and set the end-systolic elastance of the right ventricle to 43% of that for the left [See Santamore, W. P., & Burkhoff, D. (1991). Hemodynamic consequences of ventricular interaction as assessed by model analysis. The American Journal of Physiology, 260(1 Pt 2), H146-H157.] The systemic and pulmonary vessels were represented by capacitors in parallel with resistors. Pressure-sensitive diodes simulated the valves. Stressed blood volume, SBV, was defined as the total blood volume contained in the circulatory capacitors plus ventricles. As discussed in detail below, the present inventor varied systemic arterial resistance, stressed blood volume, and left ventricular passive and active properties to match hemodynamic data from the studies the present inventor simulated; all other circulatory parameters were held constant at baseline values throughout all simulations (Table 1). The present inventor implemented this model in MATLAB as a series of differential equations for changes in the volume of each compartment (left ventricle, systemic arteries, systemic veins, right ventricle, pulmonary arteries, and pulmonary veins) at 5000 time points over the cardiac cycle. A 16-GB RAM, 64-bit operating system, 3.4-GHz Intel Core i7-3770 running MATLAB 2012B ran all simulations.

TABLE 1
Circulation parameters unchanged during
acute overloading or growth.

| | | |
|---|---|---|
| Cvp | pulmonary venous compliance (ml/mmHg) | 3.0 |
| Cas | systemic arterial compliance (ml/mmHg) | 1.02 |
| Cvs | systemic venous compliance (ml/mmHg) | 17.0 |
| Cap | pulmonary arterial compliance (ml/mmHg) | 2.0 |
| Rvp | pumonary venous resistance (mmHg*s/ml) | 0.015 |
| Rcs | systemic characteristic resistance (mmHg*s/ml) | 0.023 |
| Rvs | systemic venous resistance (mmHg*s/ml) | 0.015 |
| Rcp | pulmonary characteristic resistance (mmHg*s/ml) | 0.06 |
| Rap | pulmonary arterial resistance (mmHg*s/ml) | 0.3 |

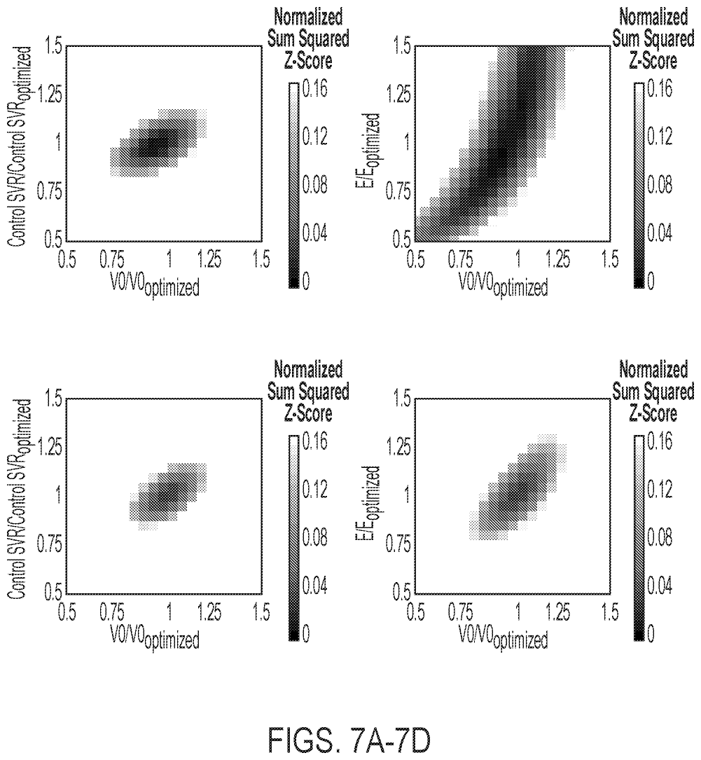

FIG. 7 visually represents the sensitivity analysis of tuned hemodynamic parameter sets against previously published experimental data [See Kleaveland, J. P., Kussmaul, W. G., Vinciguerra, T., Diters, R., & Carabello, B. A. (1988). Volume overload hypertrophy in a closedchest model of mitral regurgitation. The American Journal of Physiology, 254(6 Pt 2), H1034-H1041] and literature regarding the physiologic values of end-systolic stretch relative to an unloaded state [, See Witzenburg, C. M., & Holmes, J. W. (2017). A comparison of phenomenologic growth laws for myocardial hypertrophy. Journal of Elasticity, 129(1-2), 257-281]. FIG. 7A visually represents mean squared error (MSE) in Z scores computed as the unloaded volume of the ventricle (V₀) and systemic vascular resistance (SVR_{control}) were varied between 50 and 150% of their optimized values with all other parameters set to their optimized values and where changes in either parameter greater than 15% pushed MSE above 0.04. FIG. 7B visually represents MSE computed as V₀ and end-systolic elastance of the ventricle (E) were varied in the same manner and where many combinations of V₀ and E produced MSE values below 0.04. FIG. 7C-D visually represents that repeating the simulations from FIGS. 7A-B using an augmented objective function provided a unique optimum for all parameters including V₀and E.

The present inventor tuned the circulatory parameters in the model to match hemodynamics and LV dimensions reported at baseline and immediately following the induction of aortic constriction, mitral regurgitation, or myocardial infarction (acute). Reported heart rate and infarct size were prescribed directly. The present inventor assumed acute hemodynamic perturbation did not change the end-diastolic or end-systolic LV pressure-volume relationships and therefore, only one set of unknown LV parameters (A, B, V₀, and E) was required for each study. In contrast, the present inventor assumed that systemic vascular resistance and stressed blood volume could vary rapidly following creation of hemodynamic overload, treating both baseline and acute values for these parameters as unknowns (SVR_{control}, SVR_{acute}, SBV_{control} and SBV_{acute}). For volume overload studies, an additional unknown parameter controlling the severity of mitral valve regurgitation (MVBR_{acute}) was also determined. Thus, tuning baseline and acute hemodynamics for each study considered required identifying 8 or 9 (for volume overload) parameters.

Table 2 shows the control and acute hemodynamic data reported by each study (mean ±standard deviation); results highlighted in solid-lines-forward slash were used to tune circulatory model parameters. In general, end-diastolic and end-systolic pressures and volumes in the baseline and acutely overloaded states (EDP_{control}, ESP_{control}, EDV_{control}, ESV_{control}, EDP_{acute}, ESP_{acute}, EDV_{acute}, ESV_{acute}, n=8 values)—plus the measured regurgitant fraction for volume overload studies—should provide enough information to tune the 8-9 unknown parameters specified in the previous paragraph. Still referring to Table 2, model results and experimental data for all studies. Highlighted model values were used to tune control and acute circulatory parameters (solid-lines-forward slash), match unloaded thickness (crosshatch), or fit growth parameters (dashed-lines-backward slash). An asterisk indicates model predictions more than one standard deviation away from the reported mean. However, no study reported exactly these volumes and pressures. All studies reported measurements that could be used to estimate LV end-diastolic pressure (end-diastolic pressure, pulmonary capillary wedge pressure, or mean left atrial pressure), some measurement of blood pressure during systole (maximum LV pressure, mean arterial pressure, or systolic blood pressure), and some measurement of LV volume during systole (end-systolic LV volume, minimum LV volume, or stroke volume). Except for Nagatomo et al. [See Nagatomo, Y., Carabello, B. A., Hamawaki, M., Nemoto, S., Matsuo, T., & McDermott, P. J. (1999). Translational mechanisms accelerate the rate of protein synthesis during canine pressure-overload hypertrophy. The American Journal of Physiology - Heart and Circulatory Physiology, 277(6 Pt 2), H2176-H2184.], all studies also reported maximum LV volume or dimensions. Since Nakano et al. [See Nakano, K., Swindle, M. M., Spinale, F., Ishihara, K., Kanazawa, S., Smith, A., et al. (1991). Depressed contractile function due to canine mitral regurgitation improves after correction of the volume overload. The Journal of Clinical Investigation, 87(6), 2077-2086] and Nagatomo et al. reported similar stroke volumes, the end-diastolic pressure volume relationship from Nakano was used for both.

The present inventor estimated model parameters simultaneously using the fminsearch function in MATLAB. Differences between measured and predicted hemodynamic values were normalized by the reported standard deviation to compute Z scores, and mean squared error (MSE) in Z score was minimized. In order to reduce the complexity of the optimization, for any choice of V₀ the diastolic parameters A and B were computed directly from end-diastolic pressure-volume data (Equation 1) (discussed above with FIG. 6) prior to optimization.

For each study, E, SBV_{control}, and SBV_{acute} were initially set to values reported by Santamore and Burkhoff [See Santamore, W. P., & Burkhoff, D. (1991). Hemodynamic consequences of ventricular interaction as assessed by model analysis. The American Journal of Physiology, 260(1 Pt 2), H146-H157], while SVR_{control} and SVR_{acute} were initialized by dividing the reported mean arterial or peak systolic pressure by the product of stroke volume and heart rate. The present inventor initialized V₀ at a value that produced an end-diastolic stretch, λ, of 1.44±0.24 relative to a completely unloaded state [See Witzenburg, C. M., & Holmes, J. W. (2017). A comparison of phenomenologic growth laws for myocardial hypertrophy. Journal of Elasticity, 129(1-2), 257-281] in our thin-walled spherical model. Finally, for volume overload studies, MVBR_{acute} was initialized at 1 mmHg*s/ml.

To explore the uniqueness of the parameter sets identified by our optimization procedure, the present inventor performed a sensitivity analysis on the estimated parameter set for the Kleaveland volume overload study [See Kleaveland, J. P., Kussmaul, W. G., Vinciguerra, T., Diters, R., & Carabello, B. A. (1988). Volume overload hypertrophy in a closedchest model of mitral regurgitation. The American Journal of Physiology, 254(6 Pt 2), H1034-H1041]. The present inventor systematically varied each parameter between 50-150% of its optimized value and examined the shape of the objective function projected onto every possible two-parameter plane (FIG. 6).

At MSE values below 0.04 (darkest colors in panels A and B), hemodynamic parameters were within an average of 0.2 standard deviations of their reported mean; in most cases, variations of any one parameter of more than 15% pushed the objective function outside this range. For example, FIG. 6A shows similar dependence of MSE on V₀ and SVR_{control}, with relatively few combinations of the two parameters that hold it below 0.04. Plots of the solution space for all other model parameter pairs had a similar shape except for V₀ and E (FIG. 6B). Here, many different combinations of values yielded similarly low error. As a quantitative reflection of this error landscape analysis, the present inventor computed the area of each graph with MSE values less than 0.04. For the V₀ and E pair the area was 0.16, whereas the average for all other parameter pairs was 0.04 ±0.04 (Table 3). The present inventor concluded from this analysis that additional information or constraints are necessary in order to obtain unique, repeatable values for all parameters including V₀ and E.

The present inventor therefore augmented our objective function using additional data from the literature regarding the physiologic values of end-diastolic stretch relative to an unloaded state [See Witzenburg, C. M., & Holmes, J. W. (2017). A comparison of phenomenologic growth laws for myocardial hypertrophy. Journal of Elasticity, 129(1-2), 257-281], which varies with choice of V₀, and the peak rate of LV pressure rise, which varies with E. Table 4 gives the modified objective functions; the present inventor chose to give these literature-based terms half the weight of the data terms. Referring to Table 4, the augmented objective functions were used to customize circulatory parameters for control and acute conditions both for studies used to fit and validate growth parameters. FIGS. 6C-D show contour plots for the same projections shown in FIGS. 6A-B using the augmented objective function; including the literature terms constrained the range of acceptable values for V₀ and E without negatively impacting identification of other parameters (Table 3). Referring to Table 3, the area (FIG. 7) of each sensitivity graph with a normalized sum squared Z score less than 0.04 when fitting circulatory parameters for control and acute conditions. Please note that the first-listed numeral listed in each box is associated with the “Objective Function with only Study-Specific Data” and the second-listed numeral listed in each box is associated with the “Objective Function with Study Specific and Literature Data”.

TABLE 3

| | E | SVR_{Control} | SVR_{Acute} | SBV_{Control} | SBV_{Acute} | MVBR_{Acute} |
|---|---|---|---|---|---|---|
| V₀ | 0.163 | 0.027 | 0.043 | 0.027 | 0.009 | 0.059 |
| | 0.011 | 0.009 | 0.016 | 0.005 | 0.005 | 0.020 |
| E | | 0.061 | 0.111 | 0.057 | 0.023 | 0.152 |
| | | 0.011 | 0.016 | 0.005 | 0.005 | 0.025 |
| SVR_{Control} | | | 0.025 | 0.020 | 0.007 | 0.050 |
| | | | 0.009 | 0.009 | 0.005 | 0.023 |
| SVR_{Acute} | | | | 0.030 | 0.011 | 0.066 |
| | | | | 0.007 | 0.007 | 0.027 |
| SBV_{Control} | | | | | 0.007 | 0.039 |
| | | | | | 0.002 | 0.011 |
| SBV_{Acute} | | | | | | 0.036 |
| | | | | | | 0.011 |

TABLE 4
(part 1 of 2):
Fitting Simulations
Pressure Overload [1]
${obj}_{func} = {( \frac{{max P}_{control , model} - {max P}_{control , paper}}{{SD max P}_{control , paper}} )}^{2} + {( \frac{{max P}_{acute , model} - {max P}_{acute , paper}}{{SD max P}_{acute , paper}} )}^{2} + {( \frac{{EDP}_{control , model} - {EDP}_{control , paper}}{{SDEDP}_{control , paper}} )}^{2} + {( \frac{{EDP}_{acute , model} - {EDP}_{acute , paper}}{{SDEDP}_{acute , paper}} )}^{2} + {( \frac{{ESV}_{control , model} - {ESV}_{control , paper}}{{SDESV}_{control , paper}} )}^{2} + {( \frac{{ESV}_{acute , model} - {ESV}_{acute , paper}}{{SDESV}_{acute , paper}} )}^{2} + {( \frac{max dP \text{/} {dt}_{control , model} - max dP \text{/} {dt}_{control , sasayama}}{SD max dP \text{/} {dt}_{control , sasayama}} )}^{2} + {( \frac{λ {ED}_{control , model} - λ {ED}_{control , witzenburg}}{SD λ {ED}_{control , witzenburg}} )}^{2}$
Volume Overload [2]
${obj}_{func} = {( \frac{{MAP}_{control , model} - {MAP}_{control , paper}}{{SDMAP}_{control , paper}} )}^{2} + {( \frac{{MAP}_{acute , model} - {MAP}_{acute , paper}}{{SDMAP}_{acute , paper}} )}^{2} + {( \frac{{EDP}_{control , model} - {EDP}_{control , paper}}{{SDEDP}_{control , paper}} )}^{2} + {( \frac{{EDP}_{acute , model} - {EDP}_{acute , paper}}{{SDEDP}_{acute , paper}} )}^{2} + {( \frac{{min V}_{control , model} - {min V}_{control , paper}}{{SD min V}_{control , paper}} )}^{2} + {( \frac{{min V}_{acute , model} - {min V}_{acute , paper}}{{SD min V}_{acute , paper}} )}^{2} + {( \frac{{RF}_{acute , model} - {RF}_{acute , paper}}{{SDRF}_{acute , paper}} )}^{2} + {( \frac{max dP \text{/} {dt}_{control , model} - max dP \text{/} {dt}_{control , sasayama}}{SD max dP \text{/} {dt}_{control , sasayama}} )}^{2} + {( \frac{λ {ED}_{control , model} - λ {ED}_{control , witzenburg}}{SD λ {ED}_{control , witzenburg}} )}^{2}$
Validation Simulations
Pressure Overload [3]
${obj}_{func} = {( \frac{{max P}_{control , model} - {max P}_{control , paper}}{{SD max P}_{control , paper}} )}^{2} + {( \frac{{max P}_{acute , model} - {max P}_{acute , paper}}{{SD max P}_{acute , paper}} )}^{2} + {( \frac{{EDP}_{control , model} - {EDP}_{control , paper}}{{SDEDP}_{control , paper}} )}^{2} + {( \frac{{EDP}_{acute , model} - {EDP}_{acute , paper}}{{SDEDP}_{acute , paper}} )}^{2} + {( \frac{{SV}_{control , model} - {SV}_{control , paper}}{{SDSV}_{control , paper}} )}^{2} + {( \frac{{SV}_{acute , model} - {SV}_{acute , paper}}{{SDSV}_{acute , paper}} )}^{2} + {( \frac{max dP \text{/} {dt}_{control , model} - max dP \text{/} {dt}_{control , sasayama}}{SD max dP \text{/} {dt}_{control , sasayama}} )}^{2} + {( \frac{λ {ED}_{control , model} - λ {ED}_{control , witzenburg}}{SD λ {ED}_{control , witzenburg}} )}^{2}$
Volume Overload [4]
${obj}_{func} = {( \frac{{systolicP}_{control , model} - {systolicP}_{control , paper}}{{SDsystolicP}_{control , paper}} )}^{2} + {( \frac{{systolicP}_{acute , model} - {systolicP}_{acute , paper}}{{SDsystolicP}_{acute , paper}} )}^{2} + {( \frac{{EDP}_{control , model} - {EDP}_{control , paper}}{{SDEDP}_{control , paper}} )}^{2} + {( \frac{{EDP}_{acute , model} - {EDP}_{acute , paper}}{{SDEDP}_{acute , paper}} )}^{2} + {( \frac{{EF}_{control , model} - {EF}_{control , paper}}{{SDEF}_{control , paper}} )}^{2} + {( \frac{{EF}_{acute , model} - {EF}_{acute , paper}}{{SDEF}_{acute , paper}} )}^{2} + {( \frac{{RF}_{acute , model} - {RF}_{acute , paper}}{{SDRF}_{acute , paper}} )}^{2} + {( \frac{max dP \text{/} {dt}_{control , model} - max dP \text{/} {dt}_{control , sasayama}}{SD max dP \text{/} {dt}_{control , sasayama}} )}^{2} + {( \frac{λ {ED}_{control , model} - λ {ED}_{control , witzenburg}}{SD λ {ED}_{control , witzenburg}} )}^{2}$
Myocardial Infarction [5]
${obj}_{func} = {( \frac{{MAP}_{control , model} - {MAP}_{control , paper}}{{SDMAP}_{control , paper}} )}^{2} + {( \frac{{MAP}_{acute , model} - {MAP}_{acute , paper}}{{SDMAP}_{acute , paper}} )}^{2} + {( \frac{{EDP}_{control , model} - {EDP}_{control , paper}}{{SDEDP}_{control , paper}} )}^{2} + {( \frac{{EDP}_{acute , model} - {EDP}_{acute , paper}}{{SDEDP}_{acute , paper}} )}^{2} + {( \frac{{ESV}_{control , model} - {ESV}_{control , paper}}{{SDESV}_{control , paper}} )}^{2} + {( \frac{max dP \text{/} {dt}_{control , model} - max dP \text{/} {dt}_{control , sasayama}}{SD max dP \text{/} {dt}_{control , sasayama}} )}^{2} + {( \frac{λ {ED}_{control , model} - λ {ED}_{control , witzenburg}}{SD λ {ED}_{control , witzenburg}} )}^{2}$

FIG. 13 illustrates a method and/or system for rapidly predicting cardiac response to a heart condition and treatment strategy of a subject using a compartmental model 101 wherein said systemic and pulmonary circulations are represented as a system of resistors and capacitors 141 and said chambers of the heart are represented as spheres or assemblies of spheres (or substantially spherical or assemblies of substantially spherical shapes) or other analytic equations that relate pressure and volume to stress and strain 143. An aspect an embodiment of the present invention then calibrates the compartmental model based on disease-specific data 104.

An aspect an embodiment of the present invention models the mechanics of the left ventricular across a range of overload states using a time-varying elastance compartmental model connected to a circuit model of the circulation. Strains may be estimated from LV compartmental volumes assuming a simple spherical or substantially spherical geometry.

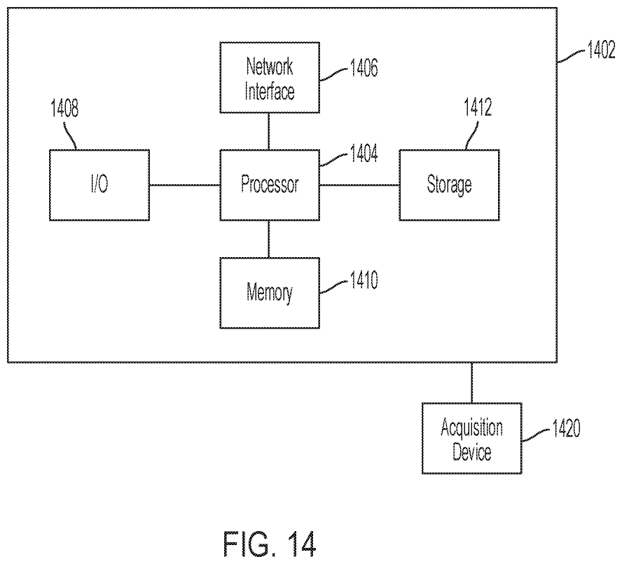

FIG. 14 is a high-level block diagram of a computer (or other machine) capable of implementing an aspect of embodiment of the present invention. The above-described methods provides, for example, rapidly predicting cardiac response to a heart condition and treatment strategy can be implemented on a computer (or machine) using well-known computer processors, memory units, storage devices, computer software, and other components. A high-level block diagram of such a computer (or machine) is illustrated in FIG. 14. Computer 1402 contains a processor 1404, which controls the overall operation of the computer 1402 (or machine) by executing computer program instructions which define such operation. The computer program instructions may be stored in a storage device 1412 (e.g., magnetic disk) and loaded into memory 1410 when execution of the computer program instructions is desired. Thus, the steps of the methods or modules of the systems (or modules of software) of FIGS. 1-4 and 13 may be defined by the computer program instructions stored in the memory 1410 and/or storage 1412 and controlled by the processor 1404 executing the computer program instructions. An acquisition device 1420, such as an image-related device or other diagnostic-related data. An image acquisition device includes at least one or more of any combination of the following: magnetic resonance imaging (MRI), ultrasound, computed tomography (CT), positron emission tomography (PET), electroanatomic mapping device, or nuclear imaging. A diagnostic device may include an electrocardiogram (ECG or EKG), other cardiac electrical data device, or diagnostic devices. The acquisition device 1420 can be connected to the computer 1402 to input image data or diagnostic data to the computer 1402. It is possible to implement the acquisition device 1420 and the computer 1402 as one device. It is also possible that the acquisition device 1420 and the computer 1402 communicate wirelessly through a network. The computer 1402 also includes one or more network interfaces 1406 for communicating with other devices via a network. The computer 1402 also includes other input/output devices 1408 that enable user interaction with the computer 1402 (e.g., display, keyboard, mouse, speakers, buttons, etc.).

Such input/output devices 1408 may be used in conjunction with a set of computer programs as an annotation tool to annotate volumes received from the acquisition device 1420. One skilled in the art will recognize that an implementation of an actual computer could contain other components as well, and that FIG. 14 is a high level representation of some of the components of such a computer (or machine) for illustrative purposes.

### EXAMPLES

Practice of an aspect of an embodiment (or embodiments) of the invention will be still more fully understood from the following examples and experimental results, which are presented herein for illustration only and should not be construed as limiting the invention in any way.

### Example and Experimental Results Set No. 1

### Examples of Potential Uses: Prognosis and Decision Support

There are many situations in cardiology and cardiothoracic surgery where the risk of performing a surgery or procedure must be weighed against the risk of waiting. In many of these situations, waiting has the potential to increase the likelihood of adverse remodeling (defined as additional heart growth and remodeling that worsens the function of the heart or complicates the planned surgery in other ways). However, no current method provides a quantitative prediction of this risk in individual patients. In these cases, an aspect of an embodiment of the present invention model could be used to, among other things, predict the expected growth and remodeling for an individual patient, valuable new information not currently available to physicians when making treatment decisions. Specific examples include:

1) Surgical repair of congenital heart disease in children. Because devices used to repair the heart do not grow as the child continues to grow, children with congenital heart disease often undergo multiple surgeries (“revisions”) to replace implanted devices with bigger devices as they grow. Waiting longer to perform each surgery can delay or reduce the number of revision surgeries, but must be weighed against the currently unknown risk of adverse remodeling. An aspect of an embodiment of the present invention model could be used to provide, among other things, patient-specific predictions of the expected adverse remodeling over a given time frame, information physicians can use to help decide when to operate.

2) Elderly or high-risk patients with valve disease. The risk of surgery to repair or replace a malfunctioning heart valve increases with age and with other factors such as coronary artery disease. These risks must be weighed against the risk of not repairing the valve, which could allow adverse remodeling of the heart that depresses heart function. By projecting the expected rate of growth and remodeling and the resulting changes in heart function, an aspect of an embodiment of the present invention model could be used to, among other things, help clinicians decide whether an individual patient's heart will remodel fast enough to justify the risk of surgery.

### Example and Experimental Results Set No. 2

### Examples of Potential Uses: Planning and Optimizing Treatment

In other situations, treatments intended to reduce or even reverse adverse remodeling of the heart have features that could be optimized if there were a way to predict how adjustments in the treatment would change future growth and remodeling of the heart. An aspect of an embodiment of the present invention model could be used to, among other things, predict future growth and remodeling for various treatment options, allowing physicians to simulate multiple options prior to a treatment in order to select the option with the best predicted response. Examples include, but not limited thereto, the following:

A) Cardiac Resynchronization Therapy (CRT). In many heart failure patients, abnormal timing of contraction in different regions of the heart (dyssynchrony) reduces the heart's ability to pump blood and causes adverse remodeling of the heart over time. CRT is a treatment in which cardiologists implanted pacemaker leads and use local stimulation by the pacemaker in one or more locations to change the pattern of electrical activation of the heart in an attempt to restore synchrony of contraction. The primary desired outcome of CRT is to reverse adverse remodeling of the heart (defined as restoring a more normal geometry, and in particular reducing end-diastolic and end-systolic volumes), but individual responses are variable, with many patients showing little or no reverse remodeling. Many features of CRT could be varied in individual patients to improve the extent of reverse remodeling, including but not limited to the choice of where to implant each lead and when to stimulate at each location. However, no method currently exists to predict how these variations will affect the reverse remodeling in a given patient. An aspect of an embodiment of the present invention model could be used to, among other things, predict the expected reverse remodeling for many different combinations of lead location, timing of stimulation, and other variables, allowing the physician to select the variations that provide the best expected outcome.

B) Noninvasive valve repair procedures. There are an increasing number of available treatments and procedures that aim to repair rather than replace damaged heart valves. As one example, the MitraClip can reduce mitral regurgitation (defined as abnormal flow of blood in the wrong direction through a valve). In many cases including the MitraClip, the treating physician has the option to apply multiple treatment steps or repetitions (e.g., applying multiple clips) depending on the cumulative effect of prior steps. There is currently no method to predict whether the current level of improvement in valve function in a given patient is sufficient to prevent adverse remodeling in the future. An aspect of an embodiment of the present invention model could be used to, among other things, predict future heart growth and remodeling for specific patients at an intermediate step of treatment, helping to decide if additional treatment steps are necessary.

### Example and Experimental Results Set No. 3

### Introduction

In the US alone, over 5 million patients suffer from heart failure, a number that is projected to exceed 8 million by 2030 [See Mozaffarian et al., Circulation, 113:e38-e360, 2016]. Heart failure increases the likelihood of conduction abnormalities such as left bundle branch block (LBBB), which causes uncoordinated contraction and dilation of the left ventricle, leading to reduced pump efficiency [See Vernooy et al., Eur Heart J, 26(1): 91-98, 2005] [See Bilchick et al., J Am Coll Cardiol, 63(16): 1657-1666, 2014]. [See Bilchick et al., J Am Coll Cardiol, 63(16): 1657-1666, 2014].

In the last two decades, cardiac resynchronization therapy (CRT) has emerged as a revolutionary therapy for patients with heart failure and LBBB. A CRT pacing device can restore coordinated contraction of the heart by electrically stimulating multiple locations at appropriate times. When it works, CRT can stop and even reverse the progression of heart failure, reducing the ventricle size and improving pump function. However, over 35% of patients still fail to respond to CRT [See Chung et al., Circulation, 117: 2608-2616, 2008]. One of the greatest strengths of CRT is that it can be customized to individual patients, offering the potential to improve patient response rates [See Bilchick et al., J Am Coll Cardiol, 63(16): 1657-1666, 2014]. Yet this also presents a dilemma: there are far too many possible lead locations and pacing settings to test directly during the implantation surgery.

Computational models can address this challenge by rapidly screening many pacing options before the CRT device implantation takes place. These models could in theory be used to pre-identify pacing locations which lead not only to the best electrical and mechanical synchrony but also to the greatest long-term reduction in ventricular volume. Several finite element models have been published that are capable of predicting cardiac growth, even in response to LBBB [See Kerckhoffs et al., Europace, 14: v65-v72, 2012]. However, these models are computationally expensive, making them impractical for routine clinical use.

In this study, the present inventor propose a computational framework (e.g., method, system, and computer readable medium) that can provide fast, patient-specific predictions of cardiac growth after the onset of LBBB. Furthermore, the present inventor demonstrates that our model's initial growth predictions agree with previously published experimental data.

### Methods

### Model of the Heart and Circulation

Mechanics of the left ventricle (LV) were modeled using a recently published compartmental model that was coupled to a circuit model of the circulation to simulate hemodynamics throughout the cardiac cycle [See Witzenburg et al., J Cardiovasc Trans Res, 11(2): 109-122, 2018]. A previously published active contraction model [See Kerckhoffs et al., J Eng Math, 47: 201-216, 2003] was adapted for use in the compartmental model, while the passive material behavior was governed by an exponential relationship between LV pressure and volumetric strain. The parameters of the active and passive LV mechanical behavior were fitted to the average pressure-volume relationship of canine hearts studied previously by our laboratory [See Fomovsky et al., Circ Heart Fail, 5(4): 515-522, 2012].

### Simulation of LBBB

The healthy LV wall contracts almost simultaneously, however LBBB is characterized by dyssynchronous mechanical activation of the LV wall [See Bilchick et al., J Am Coll Cardiol, 63(16): 1657-1666, 2014] [See Auger et al., J Magn Reson Imaging, 46(3): 887-896, 2017]. Therefore, the LV was functionally split into 10 compartments [See Sunagawa et al., Circ Res, 52(2): 170-178, 1983]. All compartments shared the same pressure at any time throughout the cardiac cycle, while the compartment volumes summed to the total LV volume. This approach allowed the present inventor to set the time of onset of mechanical activation in each compartment, thus simulating LBBB. To determine the activation times for the simulations reported here, the present inventor used DENSE MRI [See Auger et al., J Magn Reson Imaging, 46(3): 887-896, 2017] to measure the local time of onset for circumferential shortening throughout the LV wall in a dog one week after inducing LBBB using radiofrequency ablation. Baseline activation times were obtained from [See Auger et al., J Magn Reson Imaging, 46(3): 887-896, 2017].

### Strain-Driven Cardiac Growth Law

Cardiac growth was modeled by a published strain-based kinematic growth relation that allows for independent growth in the circumferential and radial direction [See Kerckhoffs et al., Mech Res Commun, 42: 40-50, 2012]. This growth law was previously adapted for use in the compartmental model and calibrated based on experimental canine pressure and volume overload studies [See Witzenburg et al., J Cardiovasc Trans Res, 11(2): 109-122, 2018]. In brief, in each LV compartment, circumferential and radial strains were estimated from volumes for each compartment by assuming a thin-walled spherical geometry, and used to drive growth of the respective compartments. Dilation was driven by a change relative to baseline of the maximum circumferential strain during the cardiac cycle, whereas thickening was driven by a change of the maximum radial strain. No changes to the published growth parameters were made for this study.

### Experimental Data Comparison

To validate the present inventor's model, the present inventor compared its results to experimental data published by Vernooy et al. [See Vernooy et al., Eur Heart J, 26(1): 91-98, 2005] who used radio frequency ablation to induce LBBB in dogs, and performed echocardiography measurements at baseline and every two weeks for 16 weeks after LBBB onset. These measurements were used to obtain changes in LV end-diastolic volumes (EDV) and wall volume at end-diastole. For comparison, the present inventor simulated 16 weeks of cardiac growth after the onset of LBBB in its fast computational model and predicted changes in LV EDV and wall volume, as well as pressure-volume loops.

### Results

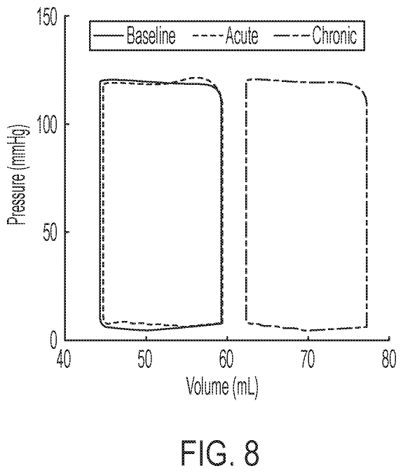

FIG. 8 graphically represents the pressure-volume relationship of the LV remained similar to baseline and acutely thereafter the onset of LBBB, but shifted to the right following growth to chronic state. 16 weeks of strain-driven cardiac growth after the onset of LBBB were simulated in just under two minutes on a laptop computer. The present inventor's model results showed that, immediately after simulating LBBB, the pressure-volume loop of the total LV was similar to baseline (FIG. 8). However, the changes in strain caused by LBBB led to increases in peak circumferential strain in many of the compartments and consequently caused cardiac growth (not shown). After 16 weeks of cardiac growth the pressure-volume loop shifted to the right.

FIG. 9A graphically represents the predicted increases (as represented by the green lines) in LV EDV versus time. FIG. 9B graphically represents the wall volume, as represent by wall volume change in percentage, (as represented by the green lines) fell well within the range of the standard deviation of previously published experimental data [See Vernooy et al., Eur Heart J, 26(1): 91-98, 2005]. Dilation was most pronounced in the latest activated compartments and was not accompanied by thickening. Without calibrating any of the growth parameters, the model-predicted evolution of EDV closely matched the experimental results (FIG. 9A). The change in LV wall volume at end diastole fell well within the standard deviation of the experimental results (FIG. 9B).

### Discussion

The goal of this study was to develop and test a fast computational framework to predict cardiac growth during ventricular dyssynchrony. The initial results of this model matched previously reported experimental results. Strikingly, this was achieved without calibrating any of the growth parameters, instead using parameters that were previously fitted to pressure and volume overload data [See Witzenburg et al., J Cardiovasc Trans Res, 11(2): 109-122, 2018]. In contrast, a previously published anatomically realistic finite-element model was only able to correctly capture growth during LBBB after changing hemodynamic parameters [See Kerckhoffs et al., Europace, 14: v65-v72, 2012]. Moreover, the finite-element model was required to run for 3 weeks on a cluster with 12 6-core processors to simulate 4 weeks of growth [See Kerckhoffs et al., Europace, 14: v65-v72, 2012] [See Witzenburg et al., J Cardiovasc Trans Res, 11(2): 109-122, 2018] whereas our model simulated 16 weeks of cardiac growth in just under two minutes on a laptop computer, making it suitable for routine clinical use.

The current model still includes several limitations. First, wall thickening was not observed in our model, but is known to occur in late-activated regions of the LV wall during LBBB. This probably caused the underestimation of the wall volume increase shown in FIG. 9B. Second, parameter sensitivity studies suggest that the choice of activation pattern and passive material properties of the myocardium strongly affect strain and therefore growth, which in our model is driven by strain. Third, for the present study the activation pattern of a single dog was obtained and simulated and compared to mean results from a separate study. Additional subject-specific, matched activation patterns and growth outcomes should be used to further test the model.

In conclusion, in the present study the present inventor demonstrated, among things, that cardiac growth, in particular LV dilation, during dyssynchrony can be predicted using a fast computational model. While this work represents just the first step towards predicting patient-specific CRT responses, the present inventor believes both the results and the time frame required to customize and run this model suggest promise for this approach in a clinical setting.

### Example and Experimental Results Set No. 4

### Introduction

The 5 million Americans who currently suffer from heart failure [See Heidenreich et al., Circ. Hear. Fail., 6(3): 606-619, 2013] are at increased risk (˜30%) of developing left bundle branch block (LBBB)]. LBBB causes uncoordinated contraction in heart failure, which can result in further left ventricular (LV) enlargement and dysfunction, leading to increasing mortality [See Baldasseroni et al., Am. Heart J., 143(3): 398-405, 2002]. Cardiac resynchronization therapy (CRT) has emerged as a revolutionary therapy for these patients. CRT can restore coordinated contraction of the heart through biventricular pacing, which can stop and even reverse the progression of heart failure. Although many patients experience favorable LV remodeling and clinical improvement with CRT [See St. John Sutton et al., Circulation, 107(15): 1985-1990, 2003] 30-50% do not have the desired response to this therapy [See Brignole et al., Eur. Heart J., 34(29): 2281-2329, 2013].

Long-term remodeling from stimulation at any one pacing location varies significantly from patient to patient, and is significantly influenced by tissue characteristics at the LV pacing site, including mechanical activation and presence of scar [See Bilchick et al., J. Am. Coll. Cardiol., 63(16): 1657-1666, 2014]. These observations suggest that optimizing pacing sites for individual patients could improve outcomes; however, there are too many possible pacing sites and settings to test in real-time during the implantation procedure. Therefore, there is a critical need for computational models that can predict outcomes (change of LV size) for various possible lead locations preoperatively.

Several finite element models have been published that are capable of predicting cardiac growth, including in response to LBBB [See Kerckhoffs et al., Europace, 14(5): v65-v72, 2012] and CRT [See Arumugamet al., Sci. Rep., 9: 2019] however, these models are computationally expensive, making them impractical for routine clinical use. The present inventor recently developed a fast compartmental model that can predict cardiac growth following volume and pressure overload [See Witzenburg et al., J. Cardiovasc. Transl. Res., 11: 109-122, 2018] as well as LBBB [See Oomen et al., SB3C 2019]. Here, the present inventors extends this framework with a fast electrical model to predict cardiac growth following CRT and demonstrate that our model's growth predictions agree with previously published experimental data.

### Methods

### Mechanical Model of the Heart and Circulation

Mechanics of the left ventricle (LV) were modeled using a recently published compartmental model that was coupled to a circuit model of the circulation to simulate hemodynamics throughout the cardiac cycle [See Witzenburg et al., J. Cardiovasc. Transl. Res., 11: 109-122, 2018]. The LV compartment was functionally divided into 16 segments according to the 16-segment AHA model. The activation timing of each segment was determined by the electrical model.

### Electrical Model of Myocardial Activation

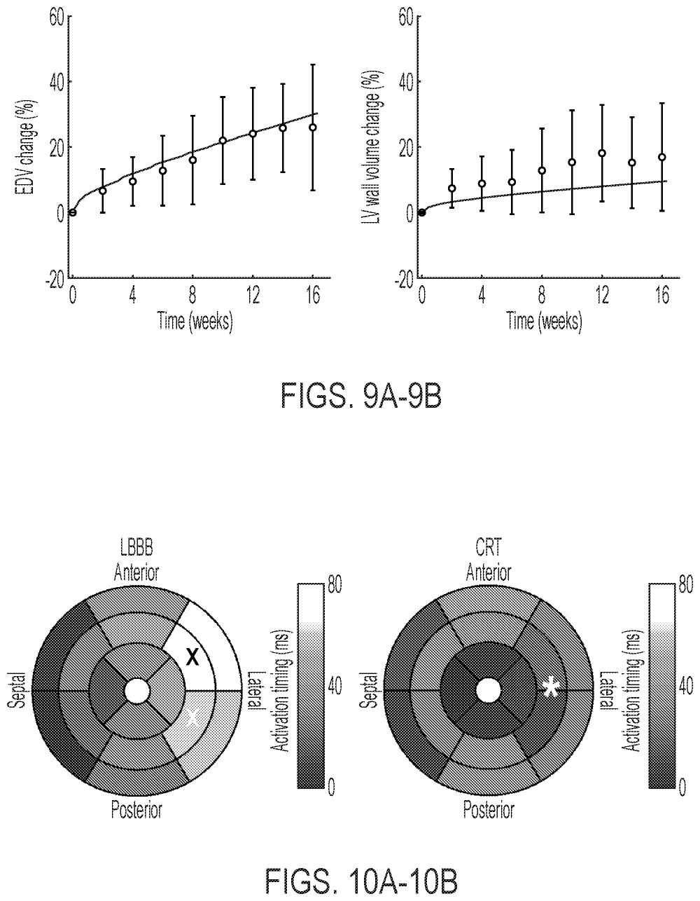

FIGS. 10A-10B graphically represents simulated activation maps of LV segments for non-ischemic for LBBB and CRT, respectively. The star (asterisk) of FIG. 10B indicates LV lead location, and the black crosses of FIG. 10A the position of simulated lateral-midwall ischemia.

The cardiac electrical activation pattern was determined using a fast graph-based method. The LBBB activation pattern was obtained by calibrating the electrical model to match 12-lead ECG data from a dog in which the present inventor induced LBBB using radiofrequency ablation. Subsequently, CRT pacing was simulated by stimulating from additional points on the RV apex and lateral LV wall. In order to couple the electrical model to the mechanical, the full-field activation timing was averaged into the 16 AHA segments (FIG. 10).

### Strain-Driven Growth Law

Cardiac growth was modeled using a strain-based volumetric isotropic growth relation similar to Kerckhoffs, et al. [See Kerckhoffs et al., Europace, 14(5): v65-v72, 2012]. The growth rate of each individual LV segment was determined by the deviation of the peak elastic circumferential strain during the current cardiac cycle E_{cc,max}ⁱ from a homeostatic setpoint. This setpoint was initially set to be equal to the maximum strain at baseline (prior to LBBB) E_{cc,max}ⁱ⁼⁰, then subsequently evolved throughout the growth simulation, a mechanism which the present inventor recently showed to be crucial for correctly predicting reversal of hypertrophy following relief of pressure overload [See Yoshida et al., Biomech. Model. Mechanobiol., 2019]. The evolving setpoint was implemented as a moving average of E_{cc,max}ⁱ, using a time window of 45 days.

### Experimental Data Comparison

To validate our model, the present inventor compared its results to experimental data published by Vernooy et al., who induced LBBB in dogs and followed them for 16 weeks [See Vernooy et al., Eur. Heart J., 28(17): 2148-2155, 2007]. After 8 weeks, CRT was started. The authors reported LV pressures and changes in LV end-diastolic volumes (EDV) and septal and lateral wall volume at end-diastole. For comparison, the present inventor simulated 16 weeks of cardiac growth after the onset of LBBB and CRT (8 weeks post-LBBB) in our fast model and predicted changes in LV EDV and regional wall volumes. Hemodynamics and growth rates were calibrated to match experimental results.

### Results

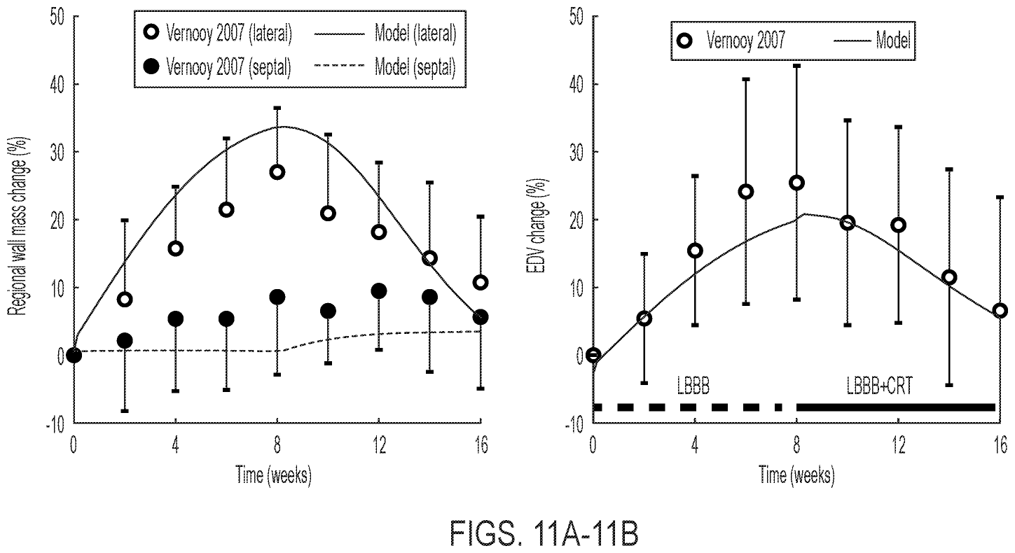

Six weeks of strain-driven cardiac growth after the onset of LBBB (FIG. 10A) and reverse growth after starting CRT (with lateral-midwall pacing, FIG. 10B) were simulated in just under two minutes on a laptop computer. FIGS. 11A-11B graphically represents the calibrated model matched changes in (a) lateral and septal wall mass (See FIG. 11A) and EDV of experimental results (See FIG. 11B) [See Vernooy et al., Eur. Heart J., 28(17): 2148-2155, 2007].

The present inventor's model results showed that the changes in strain caused by LBBB led to increases in peak circumferential strain (not shown) in the late-activated lateral wall segments and caused these segments to grow during first half of test period (FIG. 11A) . Consequently, growth of the lateral wall led to an increase of the EDV during first half of test period (FIG. 11B) during LBBB. Both predictions matched data from Vernooy et al.[See Vernooy et al., Eur. Heart J., 28(17): 2148-2155, 2007] well. Simulated CRT during the latter half of test period led to a reversal of these effects: peak strains in the lateral wall segments were reduced towards baseline due to earlier activation, causing these segment volumes to regress and thus leading to a decrease in EDV (as reflected in FIG. 11A and FIG. 11B), again in good agreement with experimental data.

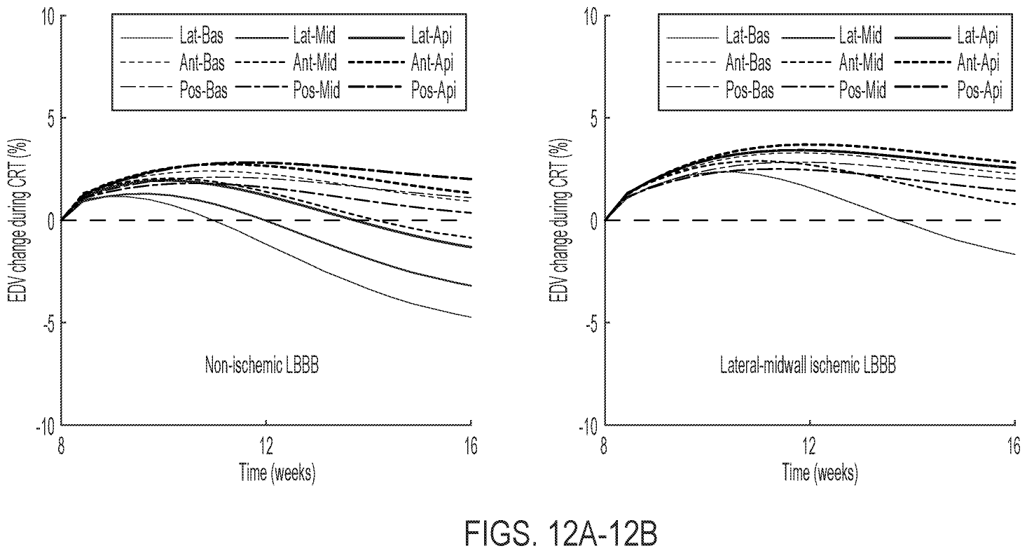

The present inventor subsequently tested the effect of different pacing locations on the change in EDV. The present inventor assumed hemodynamics remained unchanged after starting CRT to isolate the effect of lead location on CRT outcome. Both non-ischemic and ischemic LBBB were simulated, where ischemia was induced in the lateral midwall (FIG. 11) by eliminating contractility and conduction in these segments. FIGS. 12A-12B graphically represents the pacing locations influenced CRT outcomes for non-ischemia and ischemic LBBB, respectively.

The present inventor's model predicted that remodeling following CRT is dependent on lead location. For non-ischemic LBBB, pacing from the lateral-basal (Lat-Bas) segment led to the greatest reduction in EDV followed by the lateral-mid (Lat-Mid), and lateral-apical (Lat-Api) segments (as shown in FIG. 12A). Pacing from anterior (ant) and posterior (pos) segments did not lead to a reduction in EDV at 16 weeks, except for the anterior-midwall (Ant-Mid) segment. With ischemia present in the lateral-midwall (Lat-Mid, FIG. 10), the model predicted CRT would only reverse EDV below pre-CRT level when pacing from the lateral-basal segment (Lat-Bas, as shown in FIG. 12B).

### Discussion

The present inventor recently developed a fast compartmental model that can predict cardiac growth following pressure overload, volume overload, and LBBB [See Witzenburg et al., J. Cardiovasc. Transl. Res., 11: 109-122, 2018] [See Oomen et al., SB3C 2019]. In the current study, the present inventor extended this framework with a fast electrical model and reverse growth mechanism to predict remodeling following CRT. The present inventor successfully tuned our electrical and mechanical model to canine 12-lead ECG data and published experimental changes in regional wall mass and EDV. The present inventor then used its framework to investigate the differences of CRT outcome (change in EDV) dependent on pacing location. These differences became more pronounced for ischemic LBBB, which the present inventor here simulated at the lateral-midwall position. These results resemble clinical results that demonstrated CRT outcome is influenced by timing of local contraction and ischemia at the lead location [See Bilchick et al., J. Am. Coll. Cardiol., 63(16): 1657-1666, 2014].

Interestingly, reversal during CRT from an appropriate LV lead location could only be achieved when incorporating an evolving homeostatic setpoint in our mechanical model. This is consistent with recent work from Yoshida et al. [See Yoshida et al., Biomech. Model. Mechanobiol., 2019] who showed that reversal of LV volume following relief from pressure overload can only be achieved by a kinematic growth relation when incorporating an evolving setpoint.

In conclusion, in the present study the present inventor demonstrated that LV remodeling following CRT could be predicted using a fast computational model. While this work represents just the first step towards predicting patient-specific CRT responses, the present inventor believes both the results and the time frame required to customize and run this model suggest promise for this approach in a clinical setting.

### Additional Examples

Example 1. A computer-implemented method for rapidly predicting cardiac response to a heart condition and treatment strategy of a subject using a compartmental model comprising:

receiving disease-specific data;

calibrating said compartmental model based on said disease-specific data;

receiving patient-specific data;

tuning parameters using said patient-specific data;

simulating said treatment strategy using said tuned parameters with patient-specific data; and

predicting cardiac response, for use on said subject, using said simulated treatment strategy and said disease-specific-calibrated model.

Example 2. The method of example 1, further comprising:

outputting said predicted cardiac response for said use on said subject.

Example 3. The method of example 2, wherein said use on said subject of said predicted cardiac response causes a user, technician, clinician, or physician to take action on said subject based on said simulated treatment strategy.

Example 4. The method of example 1 (as well as subject matter of one or more of any combination of examples 3-26, in whole or in part), wherein said patient-specific data includes at least one or any combination of the following:

hemodynamic data; anatomic or functional imaging data from MRI, ultrasound, CT, PET, nuclear or other imaging modalities; ECG, inverse ECG, electroanatomic mapping, or any other cardiac electrical data; medical history; current and past medications; or any other patient-specific information that could affect predictions of heart responses.

Example 5. The method of example 1 (as well as subject matter of one or more of any combination of examples 2-26, in whole or in part), wherein said cardiac response includes at least one or any combination of the following:

changes in heart dimensions, mass, or cavity volumes including growth, hypertrophy, remodeling, shrinkage, or atrophy; changes in heart composition including fibrosis; and changes in heart function including improved or diminished ejection fraction, stroke work, contractility, valvular regurgitation, and synchrony or dyssnchrony of contraction.

Example 6. The method of example 1 (as well as subject matter of one or more of any combination of examples 2-26, in whole or in part), further comprising:

evaluating said predicted cardiac response to said simulated treatment strategy.

Example 7. The method of example 6 (as well as subject matter of one or more of any combination of examples 2-26, in whole or in part), further comprising:

outputting said evaluated predicted cardiac response for said use on said subject.

Example 8. The method of example 7 (as well as subject matter of one or more of any combination of examples 2-26, in whole or in part), wherein said use on said subject of said predicted cardiac response causes a user, technician, clinician, or physician to take action on said subject based on said simulated treatment strategy.

Example 9. The method of example 6 (as well as subject matter of one or more of any combination of examples 2-26, in whole or in part), further comprising:

modifying said simulated treatment strategy based on said evaluated predicted cardiac response.

Example 10. The method of example 9 (as well as subject matter of one or more of any combination of examples 2-26, in whole or in part), further comprising:

simulating said modified simulated treatment strategy using said tuned parameters with patient-specific data;

Example 11. The method of example 10 (as well as subject matter of one or more of any combination of examples 3-26, in whole or in part), further comprising:

predicting cardiac response, for use on said subject, using said modified simulated treatment strategy and said disease-specific-calibrated model.

Example 12. The method of example 1 (as well as subject matter of one or more of any combination of examples 2-26, in whole or in part), wherein said compartmental model comprises systemic and pulmonary circulations that are represented as a system of resistors and capacitors. Example 13. The method of example 12 (as well as subject matter of one or more of any combination of examples 2-26, in whole or in part), wherein said compartmental model comprises chambers of the heart that are represented using analytic equations that relate pressure and volume to stress and strain.

Example 14. The method of example 1 (as well as subject matter of one or more of any combination of examples 3-26, in whole or in part), wherein said compartmental model comprises chambers of the heart that are represented as:

spheres or assemblies of multiple spheres; or

substantially spherical shapes or assemblies of multiple substantially spherical shapes.

Example 15. The method of example 14 (as well as subject matter of one or more of any combination of examples 2-26, in whole or in part), wherein said compartmental model further comprises systemic and pulmonary circulations that are represented as a system of resistors and capacitors.

Example 16. The method of example 14 (as well as subject matter of one or more of any combination of examples 2-26, in whole or in part), wherein said compartmental model comprises chambers of the heart that are represented using analytic equations that relate pressure and volume to stress and strain.

Example 17. The method of example 1 (as well as subject matter of one or more of any combination of examples 2-26, in whole or in part), wherein said compartmental model comprises chambers of the heart that are represented using analytic equations that relate pressure and volume to stress and strain.

Example 18. The method of example 1 (as well as subject matter of one or more of any combination of examples 2-26, in whole or in part), further comprising:

generating patient-specific prognostic data from said tuned parameters.

Example 19. The method of example 18 (as well as subject matter of one or more of any combination of examples 2-26, in whole or in part), further comprising:

outputting said generated patient-specific prognostic data.

Example 20. The method of example 19 (as well as subject matter of one or more of any combination of examples 2-26, in whole or in part), wherein said patient-specific prognostic data for said use on said subject causes a user, technician, clinician, or physician to take action on said subject.

Example 21. The method of example 18 (as well as subject matter of one or more of any combination of examples 2-26, in whole or in part), further comprising:

evaluating said predicted cardiac response to said simulated treatment strategy.

Example 22. The method of example 21 (as well as subject matter of one or more of any combination of examples 2-26, in whole or in part), further comprising:

outputting said evaluated predicted cardiac response for said use on said subject.

Example 23. The method of example 22 (as well as subject matter of one or more of any combination of examples 2-26, in whole or in part), wherein said use on said subject of said predicted cardiac response causes a user, technician, clinician, or physician to take action on said subject based on said simulated treatment strategy.

Example 24. The method of example 21 (as well as subject matter of one or more of any combination of examples 2-26, in whole or in part), further comprising:

modifying said simulated treatment strategy based on said evaluated predicted cardiac response.

Example 25. The method of example 24 (as well as subject matter of one or more of any combination of examples 2-26, in whole or in part), further comprising:

simulating said modified simulated treatment strategy using said tuned parameters with patient-specific data;

Example 26. The method of example 25 (as well as subject matter of one or more of any combination of examples 2-24, in whole or in part), further comprising:

predicting cardiac response, for use on said subject, using said modified simulated treatment strategy and said disease-specific-calibrated model.

Example 27. A method for determining cardiovascular information of subject comprising:

receiving patient-specific data;

tuning parameters using said patient-specific data; and

generating patient-specific prognostic data from said tuned parameters for use on a said subject.

Example 28. The method of example 27, wherein said patient-specific prognostic data for said use on said subject causes a user, technician, clinician, or physician to take action on said subject.

Example 29. The method of example 27 (as well as subject matter of one or more of any combination of examples 28-32, in whole or in part), wherein said generated patient-specific prognostic data includes measures of contractility of undamaged myocardium following myocardial infarction, the contractility of individual subregions of the heart in the presence of electrical dyssynchrony, measures of the degree of venoconstriction; and measures of total blood volume and fluid status.

Example 30. The method of example 27 (as well as subject matter of one or more of any combination of examples 28-32, in whole or in part), wherein said generated patient-specific prognostic data includes noninvasive measures of the contractility of myocardium in any disease or condition.

Example 31. The method of example 27 (as well as subject matter of one or more of any combination of examples 28-32, in whole or in part), further comprising: outputting said generated patient-specific prognostic data.

Example 32. The method of example 31 (as well as subject matter of one or more of any combination of examples 28-31, in whole or in part), wherein said patient-specific prognostic data for said use on said subject causes a user, technician, clinician, or physician to take action on said subject.

Example 33. A system for rapidly predicting cardiac response to a heart condition and treatment strategy of a subject using a compartmental model, wherein said system comprising:

a memory storing instructions; and

a processor configured to execute the instructions to:
receive disease-specific data;
calibrate said compartmental model based on said disease-specific;
receive patient-specific data;
tune parameters using said patient-specific data;
simulate said treatment strategy using said tuned parameters with patient-specific data; and
predict cardiac response, for use on said subject, using said simulated treatment strategy and said disease-specific-calibrated model.

Example 34. The system of example 33, wherein said processor is further configured to execute the instructions to:

output said predicted cardiac response for said use on said subject.

Example 35. The system of example 34, wherein said use on said subject of said predicted cardiac response causes a user, technician, clinician, or physician to take action on said subject based on said simulated treatment strategy.

Example 36. The system of example 33 (as well as subject matter of one or more of any combination of examples 34-63, in whole or in part), wherein said patient-specific data includes at least one or any combination of the following:

hemodynamic data; anatomic or functional imaging data from MRI, ultrasound, CT, PET, nuclear or other imaging modalities; ECG, inverse ECG, electroanatomic mapping, or any other cardiac electrical data; medical history; current and past medications; or any other patient-specific information that could affect predictions of heart responses.

Example 37. The system of example 33 (as well as subject matter of one or more of any combination of examples 34-63, in whole or in part), wherein said patient-specific data is acquired from an acquisition device.

Example 38. The system of example 37 (as well as subject matter of one or more of any combination of examples 34-63, in whole or in part), wherein said acquisition device is an image acquisition device.

Example 39. The system of example 38 (as well as subject matter of one or more of any combination of examples 34-63, in whole or in part), wherein said image acquisition device includes at least one or more of any combination of the following:

magnetic resonance imaging (MRI), ultrasound, computed tomography (CT), positron emission tomography (PET), electroanatomic mapping device, or nuclear imaging.

Example 40. The system of example 33 (as well as subject matter of one or more of any combination of examples 34-63, in whole or in part), wherein said acquisition device is a diagnostic device.

Example 41. The system of example 40 (as well as subject matter of one or more of any combination of examples 34-63, in whole or in part), wherein said diagnostic acquisition device includes at least one or more of any combination of the following:

electrocardiogram (ECG or EKG) or other cardiac electrical data device.

Example 42. The system of example 33 (as well as subject matter of one or more of any combination of examples 34-63, in whole or in part), wherein said cardiac response includes at least one or any combination of the following:

changes in heart dimensions, mass, or cavity volumes including growth, hypertrophy, remodeling, shrinkage, or atrophy; changes in heart composition including fibrosis; and changes in heart function including improved or diminished ejection fraction, stroke work, contractility, valvular regurgitation, and synchrony or dyssnchrony of contraction.

Example 43. The system of example 33 (as well as subject matter of one or more of any combination of examples 34-63, in whole or in part), wherein said processor is further configured to execute the instructions to:

evaluate said predicted cardiac response to said simulated treatment strategy.

Example 44. The system of example 43 (as well as subject matter of one or more of any combination of examples 34-63, in whole or in part), wherein said processor is further configured to execute the instructions to:

output said evaluated predicted cardiac response for said use on said subject.

Example 45. The system of example 44 (as well as subject matter of one or more of any combination of examples 34-63, in whole or in part), wherein said use on said subject of said predicted cardiac response causes a user, technician, clinician, or physician to take action on said subject based on said simulated treatment strategy.

Example 46. The system of example 43 (as well as subject matter of one or more of any combination of examples 34-63, in whole or in part), wherein said processor is further configured to execute the instructions to:

modify said simulated treatment strategy based on said evaluated predicted cardiac response.

Example 47. The system of example 46 (as well as subject matter of one or more of any combination of examples 34-63, in whole or in part), further comprising:

simulate said modified simulated treatment strategy using said tuned parameters with patient-specific data;

Example 48. The system of example 47 (as well as subject matter of one or more of any combination of examples 34-63, in whole or in part), further comprising:

predict cardiac response, for use on said subject, using said modified simulated treatment strategy and said disease-specific-calibrated model.

Example 49. The system of example 33 (as well as subject matter of one or more of any combination of examples 34-63, in whole or in part), wherein said compartmental model comprises systemic and pulmonary circulations that are represented as a system of resistors and capacitors.

Example 50. The system of example 49 (as well as subject matter of one or more of any combination of examples 34-63, in whole or in part), wherein said compartmental model comprises chambers of the heart that are represented using analytic equations that relate pressure and volume to stress and strain.

Example 51. The system of example 33 (as well as subject matter of one or more of any combination of examples 34-63, in whole or in part), wherein said compartmental model comprises chambers of the heart that are represented as:

spheres or assemblies of multiple spheres; or

substantially spherical shapes or assemblies of multiple substantially spherical shapes.

Example 52. The system of example 51 (as well as subject matter of one or more of any combination of examples 34-63, in whole or in part), wherein said compartmental model further comprises systemic and pulmonary circulations that are represented as a system of resistors and capacitors.

Example 53. The system of example 51 (as well as subject matter of one or more of any combination of examples 34-63, in whole or in part), wherein said compartmental model comprises chambers of the heart that are represented using analytic equations that relate pressure and volume to stress and strain.

Example 54. The system of example 33 (as well as subject matter of one or more of any combination of examples 34-63, in whole or in part), wherein said compartmental model comprises chambers of the heart that are represented using analytic equations that relate pressure and volume to stress and strain.

Example 55. The system of example 33 (as well as subject matter of one or more of any combination of examples 34-63, in whole or in part), wherein said processor is further configured to execute the instructions to:

generate patient-specific prognostic data from said tuned parameters.

Example 56. The system of example 55 (as well as subject matter of one or more of any combination of examples 34-63, in whole or in part), wherein said processor is further configured to execute the instructions to:

output said generated patient-specific prognostic data.

Example 57. The system of example 56 (as well as subject matter of one or more of any combination of examples 34-63, in whole or in part), wherein said patient-specific prognostic data for said use on said subject causes a user, technician, clinician, or physician to take action on said subject.

Example 58. The system of example 55 (as well as subject matter of one or more of any combination of examples 34-63, in whole or in part), wherein said processor is further configured to execute the instructions to:

evaluate said predicted cardiac response to said simulated treatment strategy.

Example 59. The system of example 58 (as well as subject matter of one or more of any combination of examples 34-63, in whole or in part), wherein said processor is further configured to execute the instructions to:

output said evaluated predicted cardiac response for said use on said subject.

Example 60. The system of example 59 (as well as subject matter of one or more of any combination of examples 34-63, in whole or in part), wherein said use on said subject of said predicted cardiac response causes a user, technician, clinician, or physician to take action on said subject based on said simulated treatment strategy.

Example 61. The system of example 58, wherein said processor is further configured to execute the instructions to:

modify said simulated treatment strategy based on said evaluated predicted cardiac response.

Example 62. The system of example 61 (as well as subject matter of one or more of any combination of examples 34-63, in whole or in part), further comprising:

simulate said modified simulated treatment strategy using said tuned parameters with patient-specific data;

Example 63. The system of example 52 (as well as subject matter of one or more of any combination of examples 34-63, in whole or in part), further comprising:

predict cardiac response, for use on said subject, using said modified simulated treatment strategy and said disease-specific-calibrated model. Example 64. A system for determining cardiovascular information of subject, wherein said system comprising:

a memory storing instructions; and

a processor configured to execute the instructions to:
receive patient-specific data;
tune parameters using said patient-specific data; and
generate patient-specific prognostic data from said tuned parameters for use on a said subject.

Example 65. The system of example 64, wherein said patient-specific prognostic data for said use on said subject causes a user, technician, clinician, or physician to take action on said subject.

Example 66. The system of example 64 (as well as subject matter of one or more of any combination of examples 65-69, in whole or in part), wherein said generated patient-specific prognostic data includes measures of contractility of undamaged myocardium following myocardial infarction, the contractility of individual subregions of the heart in the presence of electrical dyssynchrony, measures of the degree of venoconstriction; and measures of total blood volume and fluid status.

Example 67. The system of example 64, wherein said generated patient-specific prognostic data includes noninvasive measures of the contractility of myocardium in any disease or condition.

68. The system of example 64 (as well as subject matter of one or more of any combination of examples 65-69, in whole or in part), wherein said processor is further configured to execute the instructions to:

output said generated patient-specific prognostic data.

Example 69. The system of example 68 (as well as subject matter of one or more of any combination of examples 65-68, in whole or in part), wherein said patient-specific prognostic data for said use on said subject causes a user, technician, clinician, or physician to take action on said subject.

Example 70. A computer program product comprising a non-transitory computer readable storage medium containing computer-executable instructions for rapidly predicting cardiac response to a heart condition and treatment strategy of a subject using a compartmental model, said instructions causing a computer to:

receive disease-specific data;

calibrate said compartmental model based on said disease-specific;

receive patient-specific data;

tune parameters using said patient-specific data;

simulate said treatment strategy using said tuned parameters with patient-specific data; and

predict cardiac response, for use on said subject, using said simulated treatment strategy and said disease-specific-calibrated model.

Example 71. The computer program product of example 70, wherein said processor is further configured to execute the instructions to:

output said predicted cardiac response for said use on said subject.

Example 72. The computer program product of example 71, wherein said use on said subject of said predicted cardiac response causes a user, technician, clinician, or physician to take action on said subject based on said simulated treatment strategy.

Example 73. The computer program product of example 70 (as well as subject matter of one or more of any combination of examples 71-100, in whole or in part), wherein said patient-specific data includes at least one or any combination of the following:

hemodynamic data; anatomic or functional imaging data from MRI, ultrasound, CT, PET, nuclear or other imaging modalities; ECG, inverse ECG, electroanatomic mapping, or any other cardiac electrical data; medical history; current and past medications; or any other patient-specific information that could affect predictions of heart responses.

Example 74. The computer program product of example 70 (as well as subject matter of one or more of any combination of examples 71-100, in whole or in part), wherein said patient-specific data is acquired from an acquisition device.

Example 75. The computer program product of example 74 (as well as subject matter of one or more of any combination of examples 71-100, in whole or in part), wherein said acquisition device is an image acquisition device.

Example 76. The computer program product of example 75 (as well as subject matter of one or more of any combination of examples 71-100, in whole or in part), wherein said image acquisition device includes at least one or more of any combination of the following:

magnetic resonance imaging (MRI), ultrasound, computed tomography (CT), positron emission tomography (PET), electroanatomic mapping device, or nuclear imaging.

Example 77. The computer program product of example 70, wherein said acquisition device is a diagnostic device.

Example 78. The computer program product of example 77 (as well as subject matter of one or more of any combination of examples 71-100, in whole or in part), wherein said diagnostic acquisition device includes at least one or more of any combination of the following:

electrocardiogram (ECG or EKG) or other cardiac electrical data device.

Example 79. The computer program product of example 70 (as well as subject matter of one or more of any combination of examples 71-100, in whole or in part), wherein said cardiac response includes at least one or any combination of the following:

changes in heart dimensions, mass, or cavity volumes including growth, hypertrophy, remodeling, shrinkage, or atrophy; changes in heart composition including fibrosis; and changes in heart function including improved or diminished ejection fraction, stroke work, contractility, valvular regurgitation, and synchrony or dyssnchrony of contraction.

Example 80. The computer program product of example 70 (as well as subject matter of one or more of any combination of examples 71-100, in whole or in part), wherein said processor is further configured to execute the instructions to:

evaluate said predicted cardiac response to said simulated treatment strategy.

Example 81. The computer program product of example 80 (as well as subject matter of one or more of any combination of examples 71-100, in whole or in part), wherein said processor is further configured to execute the instructions to:

output said evaluated predicted cardiac response for said use on said subject.

Example 82. The computer program product of example 81 (as well as subject matter of one or more of any combination of examples 71-100, in whole or in part), wherein said use on said subject of said predicted cardiac response causes a user, technician, clinician, or physician to take action on said subject based on said simulated treatment strategy.

Example 83. The computer program product of example 80 (as well as subject matter of one or more of any combination of examples 71-100, in whole or in part), wherein said processor is further configured to execute the instructions to:

modify said simulated treatment strategy based on said evaluated predicted cardiac response. Example 84. The computer program product of example 83 (as well as subject matter of one or more of any combination of examples 71-100, in whole or in part), wherein said processor is further configured to execute the instructions to:

simulate said modified simulated treatment strategy using said tuned parameters with patient-specific data;

Example 85. The computer program product of example 84 (as well as subject matter of one or more of any combination of examples 71-100, in whole or in part), further comprising:

predict cardiac response, for use on said subject, using said modified simulated treatment strategy and said disease-specific-calibrated model.

Example 86. The computer program product of example 70 (as well as subject matter of one or more of any combination of examples 71-100, in whole or in part), wherein said compartmental model comprises systemic and pulmonary circulations that are represented as a system of resistors and capacitors.

Example 87. The computer program product of example 86 (as well as subject matter of one or more of any combination of examples 71-100, in whole or in part), wherein said compartmental model comprises chambers of the heart that are represented using analytic equations that relate pressure and volume to stress and strain.

Example 88. The computer program product of example 70 (as well as subject matter of one or more of any combination of examples 71-100, in whole or in part), wherein said compartmental model comprises chambers of the heart that are represented as:

spheres or assemblies of multiple spheres; or

substantially spherical shapes or assemblies of multiple substantially spherical shapes.

Example 89. The computer program product of example 88 (as well as subject matter of one or more of any combination of examples 71-100, in whole or in part), wherein said compartmental model further comprises systemic and pulmonary circulations that are represented as a system of resistors and capacitors.

Example 90. The computer program product of example 88 (as well as subject matter of one or more of any combination of examples 71-100, in whole or in part), wherein said compartmental model comprises chambers of the heart that are represented using analytic equations that relate pressure and volume to stress and strain.

Example 91. The computer program product of example 70 (as well as subject matter of one or more of any combination of examples 71-100, in whole or in part), wherein said compartmental model comprises chambers of the heart that are represented using analytic equations that relate pressure and volume to stress and strain.

Example 92. The computer program product of example 70 (as well as subject matter of one or more of any combination of examples 71-100, in whole or in part), wherein said processor is further configured to execute the instructions to:

generate patient-specific prognostic data from said tuned parameters.

Example 93. The computer program product of example 92 (as well as subject matter of one or more of any combination of examples 71-100, in whole or in part), wherein said processor is further configured to execute the instructions to:

output said generated patient-specific prognostic data.

Example 94. The computer program product of example 93 (as well as subject matter of one or more of any combination of examples 71-100, in whole or in part), wherein said patient-specific prognostic data for said use on said subject causes a user, technician, clinician, or physician to take action on said subject. p Example 95. The computer program product of example 92 (as well as subject matter of one or more of any combination of examples 71-100, in whole or in part), wherein said processor is further configured to execute the instructions to:

evaluate said predicted cardiac response to said simulated treatment strategy.

Example 96. The computer program product of example 95 (as well as subject matter of one or more of any combination of examples 71-100, in whole or in part), wherein said processor is further configured to execute the instructions to:

output said evaluated predicted cardiac response for said use on said subject.

Example 97. The computer program product of example 96 (as well as subject matter of one or more of any combination of examples 71-100, in whole or in part), wherein said use on said subject of said predicted cardiac response causes a user, technician, clinician, or physician to take action on said subject based on said simulated treatment strategy.

Example 98. The computer program product of example 95 (as well as subject matter of one or more of any combination of examples 71-100, in whole or in part), wherein said processor is further configured to execute the instructions to:

modify said simulated treatment strategy based on said evaluated predicted cardiac response.

Example 99. The computer program product of example 98 (as well as subject matter of one or more of any combination of examples 71-100, in whole or in part), wherein said processor is further configured to execute the instructions to:

simulate said modified simulated treatment strategy using said tuned parameters with patient-specific data;

Example 100. The computer program product of example 99 (as well as subject matter of one or more of any combination of examples 71-100, in whole or in part), further comprising:

predict cardiac response, for use on said subject, using said modified simulated treatment strategy and said disease-specific-calibrated model.

Example 101. A computer program product comprising a non-transitory computer readable storage medium containing computer-executable instructions for determining cardiovascular information of subject, said instructions causing a computer to:
receive patient-specific data;
tune parameters using said patient-specific data; and
generate patient-specific prognostic data from said tuned parameters for use on a said subject.

Example 102. The computer program product of example 101, wherein said patient-specific prognostic data for said use on said subject causes a user, technician, clinician, or physician to take action on said subject.

Example 103. The computer program product of example 101 (as well as subject matter of one or more of any combination of examples 102-106, in whole or in part), wherein said generated patient-specific prognostic data includes measures of contractility of undamaged myocardium following myocardial infarction, the contractility of individual subregions of the heart in the presence of electrical dyssynchrony, measures of the degree of venoconstriction; and measures of total blood volume and fluid status.

Example 104. The computer program product of example 101 (as well as subject matter of one or more of any combination of examples 102-106, in whole or in part), wherein said generated patient-specific prognostic data includes noninvasive measures of the contractility of myocardium in any disease or condition.

Example 105. The computer program product of example 101 (as well as subject matter of one or more of any combination of examples 102-106, in whole or in part), wherein said processor is further configured to execute the instructions to:

output said generated patient-specific prognostic data.

Example 106. The computer program product of example 105 (as well as subject matter of one or more of any combination of examples 102-105, in whole or in part), wherein said patient-specific prognostic data for said use on said subject causes a user, technician, clinician, or physician to take action on said subject.

Example 107. The example of any given method associated with any single example, any combination of one or more examples, or all of the examples as provided in Examples 1-32, which can be fully performed (executed) for a duration with one or more of any combination of the following ranges:

greater than zero seconds and less than about 1 second;

greater than zero seconds and less than about two seconds;

greater than zero seconds and less than about five seconds;

greater than zero seconds and less than about ten seconds;

greater than zero seconds and less than about thirty seconds;

greater than zero seconds and less than about 1 minute;

greater than zero seconds and less than about 2 minutes;

greater than zero seconds and less than about 5 minutes;

greater than zero seconds and less than about 15 minutes;

greater than zero seconds and less than about 30 minutes;

greater than zero seconds and less than about an hour;

greater than zero seconds and less than about two hours;

greater than zero seconds and less than about four hours;

greater than zero seconds and less than about twelve hours;

greater than zero seconds and less than about twenty-four hours; or

greater than zero seconds and less than one week.

The numerical ranges recited herein by endpoints include all numbers and fractions subsumed within that range. Similarly, numerical ranges recited herein by endpoints include subranges subsumed within that range. It should be appreciated that the specified duration may be greater than twenty four hours. It should be appreciated that the specified duration may be greater one week.

Example 108. The example of any given system associated with any single example, any combination of one or more examples, or all of the examples as provided in Examples 33-69, which can be fully performed (executed) for a duration with one or more of any combination of the following ranges:

greater than zero seconds and less than about 1 second;

greater than zero seconds and less than about two seconds;

greater than zero seconds and less than about five seconds;

greater than zero seconds and less than about ten seconds;

greater than zero seconds and less than about thirty seconds;

greater than zero seconds and less than about 1 minute;

greater than zero seconds and less than about 2 minutes;

greater than zero seconds and less than about 5 minutes;

greater than zero seconds and less than about 15 minutes;

greater than zero seconds and less than about 30 minutes;

greater than zero seconds and less than about an hour;

greater than zero seconds and less than about two hours;

greater than zero seconds and less than about four hours;

greater than zero seconds and less than about twelve hours;

greater than zero seconds and less than about twenty-four hours; or

greater than zero seconds and less than one week.

The numerical ranges recited herein by endpoints include all numbers and fractions subsumed within that range. Similarly, numerical ranges recited herein by endpoints include subranges subsumed within that range. It should be appreciated that the specified duration may be greater than twenty four hours. It should be appreciated that the specified duration may be greater one week.

Example 109. The example of any given computer program product associated with any single example, any combination of one or more examples, or all of the examples as provided in Examples 70-106, which can be fully performed (executed) for a duration with one or more of any combination of the following ranges:

greater than zero seconds and less than about 1 second;

greater than zero seconds and less than about two seconds;

greater than zero seconds and less than about five seconds;

greater than zero seconds and less than about ten seconds;

greater than zero seconds and less than about thirty seconds;

greater than zero seconds and less than about 1 minute;

greater than zero seconds and less than about 2 minutes;

greater than zero seconds and less than about 5 minutes;

greater than zero seconds and less than about 15 minutes;

greater than zero seconds and less than about 30 minutes;

greater than zero seconds and less than about an hour;

greater than zero seconds and less than about two hours;

greater than zero seconds and less than about four hours;

greater than zero seconds and less than about twelve hours;

greater than zero seconds and less than about twenty-four hours; or

greater than zero seconds and less than one week.

The numerical ranges recited herein by endpoints include all numbers and fractions subsumed within that range. Similarly, numerical ranges recited herein by endpoints include subranges subsumed within that range. It should be appreciated that the specified duration may be greater than twenty four hours. It should be appreciated that the specified duration may be greater one week.

Example 110. The method of using any of the elements, components, devices, computer program product and systems or their sub-components provided in any one or more of examples 1-106, in whole or in part.

Example 111. The method of manufacturing any of the elements, components, devices, computer program product, and systems or their sub-components provided in any one or more of examples 1-106, in whole or in part.

### REFERENCES

The devices, systems, apparatuses, compositions, computer program products, non-transitory computer readable medium, networks, acquisition devices, and methods of various embodiments of the invention disclosed herein may utilize aspects (such as devices, apparatuses, systems, compositions, computer program products, non-transitory computer readable medium, networks, acquisition devices, and methods) disclosed in the following references, applications, publications and patents and which are hereby incorporated by reference herein in their entirety (and which are not admitted to be prior art with respect to the present invention by inclusion in this section):

1. U.S. Pat. No. 8,682,626 B2, Ionasec, et al., “Method and System for Comprehensive Patient-Specific Modeling of the Heart”, Mar. 25, 2014.
2. U.S. Patent Application Publication No. US 2012/0022843 A1, Ionasec, et al., “Method and System for Comprehensive Patient-Specific Modeling of the Heart, Jan. 26, 2012.
3. U.S. Patent Application Publication No. US 2007/0014452 A1, Suresh, et al., “Method and System for Image Processing and Assessment of a State of a Heart”, Jan. 18, 2007.
4. U.S. Pat. No. 7,773,785 B2, Murphy, et al., “System and Method for Facilitating Cardiac Intervention”, Aug. 10, 2010.
5. U.S. Pat. No. 7,536,042 B2, Murphy, et al., “System and Method for Facilitating Cardiac Intervention”, May 19, 2009.
6. U.S. Pat. No. 9,613,186 B2, Fonte, “Systems and Methods for Determining Blood Flow Characteristics Using Flow Ratio”, Apr. 4, 2017.
7. U.S. Patent Application Publication No. US 2016/0262635 A1, McCullouch, et al, “Compositions, Devices and Methods for Diagnosing Heart Failure and for Patient-Specific Modeling to Predict Outcomes of Cardiac Resynchonization Therapy”, Sep. 15, 2016.
8. Japanese Patent No. JP6203641 B2, Trayanova, et al, “System, Method and Program for Planning Cardiac Surgery by Patient Using a Geometric Model and 3D Imaging to Plan Surgeries”, Sep. 27, 2017.
9. U.S. Patent Application Publication No. US 2014/0088943 A1, Trayanova, et al, “System and Method for Planning a Patient-Specific Cardiac Procedure”, Mar. 27, 2014.
10. International Patent Application Publ. No. WO 2012/109618 A2, Trayanova, et al, “System and Method for Planning a Patient-Specific Cardiac Procedure”, Aug. 16, 2012.
11. U.S. Pat. No. 10,363,100 B2, Trayanova, et al, “Systems and Methods for Patient-Specific Modeling of the Heart for Prediction of Targets for Catheter Ablation of Ventricular Tachycardia in Patients with Implantable Cardioverter Defibrillators”, Jul. 30, 2019.
12. U.S. Patent Application Publication No. US 2019/0038357 A1, Adler, “Methods of Cardiac Mapping and Model Merging”, Feb. 7, 2019.
13. Japanese Patent No. JP5868052 B2, Ionasec, et al, “Comprehensive Patient-Specific Heart Modeling Method and System”, Feb. 24, 2016.
14. U.S. Pat. No. 8,682,626 B2, Ionasec, et al., “Method and System for Comprehensive Patient-Specific Modeling of the Heart”, March 25, 2014.
15. U.S. Patent Application Publication No. US 2017/0311839 A1, Osman, “Rapid Quantitative Evaluations of Heart Function with Strain Measurements from MRI”, November 2, 2017.
16. Surkova E, Badano LP, Bellu R, Aruta P, Sambugaro F, Romeo G, Migiore F, Muraru D, “Left Bundle Branch Block: From Cardiac Mechanics to Clinical and Diagnostic Challenges”, Eurospace 2017, 19: 1251-1271. Doi: 10/1093/eurospace/eux061.
17. U.S. Pat. No. 9,805,463 B2, Choi, et al, “Systems and Methods for Predicting Location, Onset, and/or Change of Coronary Lesions”, Oct. 31, 2017.
18. U.S. Pat. No. 10,096,104 B2, Choi, et al, “Systems and Methods for Predicting Location, Onset, and/or Change of Coronary Lesions”, Oct. 9, 2018.
19. U.S. Patent Application Publication No. US 2019/0019289 A1, Choi, et al, “Systems and Methods for Predicting Location, Onset, and/or Change of Coronary Lesions”, Jan. 17, 2019.
20. U.S. Patent Application Publication No. US 2006/0211909 A1, Anstadt, et al, “Method and Apparatus for Direct Mechanical Ventricular Actuation with Favorable Conditioning and Minimal Heart Stress”, Sep.21, 2006.
21. U.S. Patent Application Publication No. US 2016/0210435 A1, Neumann, et al, “Systems and Methods for Estimating Physiological Heart Measurements from Medical Images and Clinical Data”, Jul. 21, 2016.
22. Witzenburg C. M., Holmes J. W., “Predicting the Time Course of Ventricular Dilation and Thickening Using a Rapid Compartmental Model”, Journal of Cardiovascular Translational Research 2018, 11: 109-122. https://doi.org/10.1007/s12265-18-9793-1.
23. Witzenburg, C. M., Holmes, J. W. Electronic Supplemental Material for “Predicting the Time Course of Ventricular Dilation and Thickening Using a Rapid Compartmental model”, Journal of Cardiovascular Translational Research 2018, 11: 109-122. https://doi.org/10.1007/s12265-18-9793-1.
24. Oomen, P. J. A., Witzenburg, C. M., Phung, T. N., Bilchick, K. C., Holmes, J. W., “Fast Predictions of Cardiac Growth During Ventricular Dyssynchrony”, 2019 Summer Biomechanics, Bioengineering and Biotransport Conference (SB3C2019) Proceedings.
25. Savinova, O. V., & Gerdes, A. M. (2012). Myocyte changes in heart failure. Heart Failure Clinics, 8(1), 1-6.
26. O'Gara, P. T., Kushner, F. G., Ascheim, D. D., Casey, D. E., Chung, M. K., De Lemos, J. A., et al. (2013). 2013 ACCF/AHA guideline for the management of st-elevation myocardial infarction. Circulation, 127(4), e362-425.
27. Yancy, C. W., Jessup, M., Bozkurt, B., Butler, J., Casey, D. E., Drazner, M. H., et al. (2013). 2013 ACCF/AHA Guideline for the Management of Heart Failure. Circulation, 128(16), 1810-1852.
28. Gardin, J. M., Mcclelland, R., Kitzman, D., Lima, J. A. C., Bommer, W., Klopfenstein, H. S., et al. (2001). M-mode echocardiographic predictors of six- to seven-year incidence of coronary heart disease, stroke, congestive heart failure, and mortality in an elderly cohort (The Cardiovascular Health Study). The American Journal of Cardiology, 87(9), 1051-1057.
29. Aurigemma, G. P., Gottdiener, J. S., Shemanski, L., Gardin, J., & Kitzman, D. (2001). Predictive value of systolic and diastolic function for incident congestive heart failure in the elderly: the cardiovascular health study. Journal of the American College of Cardiology, 37(4), 1042-1048.
30. Nishimura, R. A., Otto, C. M., Bonow, R. O., Carabello, B. A., Erwin, J. P., Guyton, R. A., et al. (2014). 2014AHA/ACC guideline for the management of patients with valvular heart disease: executive summary. Circulation, 129(23), 2440-2492.
31. Bonow, R. O., Carabello, B. A., Chatterjee, K., de Leon, A. C., Faxon, D. P., Freed, M. D., et al. (2008). 2008. Focused Update Incorporated Into the ACC/AHA 2006 Guidelines for the Management of Patients With Valvular. Heart Disease. Circulation, 118(15), e523-e661.
32. Suri, R. M., Vanoverschelde, J., Grigioni, F., Schaff, H. V., Tribouilloy, C., Avierinos, J., et al. (2013). Association between early surgical intervention vs watchful waiting and outcomes for mitral regurgitation due to flail mitral valve leaflets. The Journal of the American Medical Association, 310(6), 609-616.
33. Feinstein, J. A., Benson, D. W., Dubin, A. M., Cohen, M. S., Maxey, D. M., Mahle, W. T., et al. (2012). Hypoplastic left heart syndrome: current considerations and expectations. Journal of the American College of Cardiology, 59(1 SUPPL), S1-S42.
34. Mozaffarian et al., Circulation, 113: e38-e360, 2016.

### 35. Vernooy et al., Eur Heart J, 26(1): 91-98, 2005].

### 36. Bilchick et al., J Am Coll Cardiol, 63(16): 1657-1666, 2014.

37. St. John Sutton et al., Circulation, 107(15): 1985-1990, 2003.
38. Brignole et al., Eur. Heart J., 34(29): 2281-2329, 2013.
39. Bilchick et al., J. Am. Coll. Cardiol., 63(16): 1657-1666, 2014.
40. Kerckhoffs et al., Europace, 14(5): v65-v72, 2012.
41. Arumugamet al., Sci. Rep., 9: 2019.
42. Kleaveland, J. P., Kussmaul, W. G., Vinciguerra, T., Diters, R., & Carabello, B. A. (1988). Volume overload hypertrophy in a closedchest model of mitral regurgitation. The American Journal of Physiology, 254(6 Pt 2), H1034-H1041.
43. Witzenburg, C. M., & Holmes, J. W. (2017). A comparison of phenomenologic growth laws for myocardial hypertrophy. Journal of Elasticity, 129(1-2), 257-281.
44. Vernooy et al., Eur. Heart J., 28(17): 2148-2155, 2007.

### 45. Chung et al.,Circulation, 117: 2608-2616, 2008.

### 46. Kerckhoffs et al., J Eng Math, 47: 201-216, 2003.

### 47. Fomovsky et al., Circ Heart Fail, 5(4): 515-522, 2012.

### 48. Auger et al., J Magn Reson Imaging, 46(3): 887-896, 2017.

### 49. Sunagawa et al., Circ Res, 52(2): 170-178, 1983.

### 50. Kerckhoffs et al., Mech Res Commun, 42: 40-50, 2012.

51. Heidenreich et al., Circ. Hear. Fail., 6(3): 606-619, 2013.
52. Baldasseroni et al., Am. Heart J., 143(3): 398-405, 2002.
53. Yoshida et al., Biomech. Model. Mechanobiol., 2019.
54. Santamore, W. P., & Burkhoff, D. (1991). Hemodynamic consequences of ventricular interaction as assessed by model analysis. The American Journal of Physiology, 260(1 Pt 2), H146-H157.

Unless clearly specified to the contrary, there is no requirement for any particular described or illustrated activity or element, any particular sequence or such activities, any particular size, speed, material, duration, contour, dimension or frequency, or any particularly interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. It should be appreciated that aspects of the present invention may have a variety of sizes, contours, shapes, compositions and materials as desired or required.

In summary, while the present invention has been described with respect to specific embodiments, many modifications, variations, alterations, substitutions, and equivalents will be apparent to those skilled in the art. The present invention is not to be limited in scope by the specific embodiment described herein. Indeed, various modifications of the present invention, in addition to those described herein, will be apparent to those of skill in the art from the foregoing description and accompanying drawings. Accordingly, the invention is to be considered as limited only by the spirit and scope of the disclosure (and claims), including all modifications and equivalents.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particularly interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

What is claimed is:

1. A computer-implemented method for rapidly predicting cardiac response to a heart condition and treatment strategy of a subject using a compartmental model comprising:
receiving disease-specific data;
calibrating said compartmental model based on said disease-specific data;
receiving patient-specific data;
tuning parameters using said patient-specific data;
simulating said treatment strategy using said tuned parameters with patient-specific data; and
predicting cardiac response, for use on said subject, using said simulated treatment strategy and said disease-specific-calibrated model.

2. The method of claim 1, further comprising:
outputting said predicted cardiac response for said use on said subject.

3. The method of claim 2, wherein said use on said subject of said predicted cardiac response causes a user, technician, clinician, or physician to take action on said subject based on said simulated treatment strategy.

4. The method of claim 1, wherein said patient-specific data includes at least one or any combination of the following:
hemodynamic data; anatomic or functional imaging data from MRI, ultrasound, CT, PET, nuclear or other imaging modalities; ECG, inverse ECG, electroanatomic mapping, or any other cardiac electrical data; medical history; current and past medications; or any other patient-specific information that could affect predictions of heart responses.

5. The method of claim 1, wherein said cardiac response includes at least one or any combination of the following:
changes in heart dimensions, mass, or cavity volumes including growth, hypertrophy, remodeling, shrinkage, or atrophy; changes in heart composition including fibrosis; and changes in heart function including improved or diminished ejection fraction, stroke work, contractility, valvular regurgitation, and synchrony or dyssnchrony of contraction.

6. The method of claim 1, further comprising:
evaluating said predicted cardiac response to said simulated treatment strategy.

7. The method of claim 6, further comprising:
outputting said evaluated predicted cardiac response for said use on said subject.

8. The method of claim 7, wherein said use on said subject of said predicted cardiac response causes a user, technician, clinician, or physician to take action on said subject based on said simulated treatment strategy.

9. The method of claim 6, further comprising:
modifying said simulated treatment strategy based on said evaluated predicted cardiac response.

10. The method of claim 9, further comprising:
simulating said modified simulated treatment strategy using said tuned parameters with patient-specific data;

11. The method of claim 10, further comprising:
predicting cardiac response, for use on said subject, using said modified simulated treatment strategy and said disease-specific-calibrated model.

12. The method of claim 1, wherein said compartmental model comprises systemic and pulmonary circulations that are represented as a system of resistors and capacitors.

13. The method of claim 12, wherein said compartmental model comprises chambers of the heart that are represented using analytic equations that relate pressure and volume to stress and strain.

14. The method of claim 1, wherein said compartmental model comprises chambers of the heart that are represented as:
spheres or assemblies of multiple spheres; or substantially spherical shapes or assemblies of multiple substantially spherical shapes.

15. The method of claim 14, wherein said compartmental model further comprises systemic and pulmonary circulations that are represented as a system of resistors and capacitors.

16. The method of claim 14, wherein said compartmental model comprises chambers of the heart that are represented using analytic equations that relate pressure and volume to stress and strain.

17. The method of claim 1, wherein said compartmental model comprises chambers of the heart that are represented using analytic equations that relate pressure and volume to stress and strain.

18. The method of claim 1, further comprising:
generating patient-specific prognostic data from said tuned parameters.

19. The method of claim 18, further comprising:
outputting said generated patient-specific prognostic data.

20. The method of claim 19, wherein said patient-specific prognostic data for said use on said subject causes a user, technician, clinician, or physician to take action on said subject.

21. The method of claim 18, further comprising:
evaluating said predicted cardiac response to said simulated treatment strategy.

22. The method of claim 21, further comprising:
outputting said evaluated predicted cardiac response for said use on said subject.

23. The method of claim 22, wherein said use on said subject of said predicted cardiac response causes a user, technician, clinician, or physician to take action on said subject based on said simulated treatment strategy.

24. The method of claim 21, further comprising:
modifying said simulated treatment strategy based on said evaluated predicted cardiac response.

25. The method of claim 24, further comprising:
simulating said modified simulated treatment strategy using said tuned parameters with patient-specific data;

26. The method of claim 25, further comprising:
predicting cardiac response, for use on said subject, using said modified simulated treatment strategy and said disease-specific-calibrated model.

27. A method for determining cardiovascular information of subject comprising:
receiving patient-specific data;
tuning parameters using said patient-specific data; and
generating patient-specific prognostic data from said tuned parameters for use on a said subject.

28. The method of claim 27, wherein said patient-specific prognostic data for said use on said subject causes a user, technician, clinician, or physician to take action on said subject.

29. The method of claim 27, wherein said generated patient-specific prognostic data includes measures of contractility of undamaged myocardium following myocardial infarction, the contractility of individual subregions of the heart in the presence of electrical dyssynchrony, measures of the degree of venoconstriction; and measures of total blood volume and fluid status.

30. The method of claim 27, wherein said generated patient-specific prognostic data includes noninvasive measures of the contractility of myocardium in any disease or condition.

31. The method of claim 27, further comprising:
outputting said generated patient-specific prognostic data.

32. The method of claim 31, wherein said patient-specific prognostic data for said use on said subject causes a user, technician, clinician, or physician to take action on said subject.

33. A system for rapidly predicting cardiac response to a heart condition and treatment strategy of a subject using a compartmental model, wherein said system comprising:
a memory storing instructions; and
a processor configured to execute the instructions to:
receive disease-specific data;
calibrate said compartmental model based on said disease-specific;
receive patient-specific data;
tune parameters using said patient-specific data;
simulate said treatment strategy using said tuned parameters with patient-specific data; and
predict cardiac response, for use on said subject, using said simulated treatment strategy and said disease-specific-calibrated model.

34. The system of claim 33, wherein said processor is further configured to execute the instructions to:
output said predicted cardiac response for said use on said subject.

35. The system of claim 34, wherein said use on said subject of said predicted cardiac response causes a user, technician, clinician, or physician to take action on said subject based on said simulated treatment strategy.

36. The system of claim 33, wherein said patient-specific data includes at least one or any combination of the following:
hemodynamic data; anatomic or functional imaging data from MRI, ultrasound, CT, PET, nuclear or other imaging modalities; ECG, inverse ECG, electroanatomic mapping, or any other cardiac electrical data; medical history; current and past medications; or any other patient-specific information that could affect predictions of heart responses.

37. The system of claim 33, wherein said patient-specific data is acquired from an acquisition device.

38. The system of claim 37, wherein said acquisition device is an image acquisition device.

39. The system of claim 38, wherein said image acquisition device includes at least one or more of any combination of the following:
magnetic resonance imaging (MRI), ultrasound, computed tomography (CT), positron emission tomography (PET), electroanatomic mapping device, or nuclear imaging.

40. The system of claim 33, wherein said acquisition device is a diagnostic device.

41. The system of claim 40, wherein said diagnostic acquisition device includes at least one or more of any combination of the following:
electrocardiogram (ECG or EKG) or other cardiac electrical data device.

42. The system of claim 33, wherein said cardiac response includes at least one or any combination of the following:
changes in heart dimensions, mass, or cavity volumes including growth, hypertrophy, remodeling, shrinkage, or atrophy; changes in heart composition including fibrosis; and changes in heart function including improved or diminished ejection fraction, stroke work, contractility, valvular regurgitation, and synchrony or dyssnchrony of contraction.

43. The system of claim 33, wherein said processor is further configured to execute the instructions to:
evaluate said predicted cardiac response to said simulated treatment strategy.

44. The system of claim 43, wherein said processor is further configured to execute the instructions to:
output said evaluated predicted cardiac response for said use on said subject.

45. The system of claim 44, wherein said use on said subject of said predicted cardiac response causes a user, technician, clinician, or physician to take action on said subject based on said simulated treatment strategy.

46. The system of claim 43, wherein said processor is further configured to execute the instructions to:
modify said simulated treatment strategy based on said evaluated predicted cardiac response.

47. The system of claim 46, further comprising:
simulate said modified simulated treatment strategy using said tuned parameters with patient-specific data;

48. The system of claim 47, further comprising:
predict cardiac response, for use on said subject, using said modified simulated treatment strategy and said disease-specific-calibrated model.

49. The system of claim 33, wherein said compartmental model comprises systemic and pulmonary circulations that are represented as a system of resistors and capacitors.

50. The system of claim 49, wherein said compartmental model comprises chambers of the heart that are represented using analytic equations that relate pressure and volume to stress and strain.

51. The system of claim 33, wherein said compartmental model comprises chambers of the heart that are represented as:
spheres or assemblies of multiple spheres; or
substantially spherical shapes or assemblies of multiple substantially spherical shapes.

52. The system of claim 51, wherein said compartmental model further comprises systemic and pulmonary circulations that are represented as a system of resistors and capacitors.

53. The system of claim 51, wherein said compartmental model comprises chambers of the heart that are represented using analytic equations that relate pressure and volume to stress and strain.

54. The system of claim 33, wherein said compartmental model comprises chambers of the heart that are represented using analytic equations that relate pressure and volume to stress and strain.

55. The system of claim 33, wherein said processor is further configured to execute the instructions to:
generate patient-specific prognostic data from said tuned parameters.

56. The system of claim 55, wherein said processor is further configured to execute the instructions to:
output said generated patient-specific prognostic data.

57. The system of claim 56, wherein said patient-specific prognostic data for said use on said subject causes a user, technician, clinician, or physician to take action on said subject.

58. The system of claim 55, wherein said processor is further configured to execute the instructions to:
evaluate said predicted cardiac response to said simulated treatment strategy.

59. The system of claim 58, wherein said processor is further configured to execute the instructions to:
output said evaluated predicted cardiac response for said use on said subject.

60. The system of claim 59, wherein said use on said subject of said predicted cardiac response causes a user, technician, clinician, or physician to take action on said subject based on said simulated treatment strategy.

61. The system of claim 58, wherein said processor is further configured to execute the instructions to:
modify said simulated treatment strategy based on said evaluated predicted cardiac response.

62. The system of claim 61, further comprising:
simulate said modified simulated treatment strategy using said tuned parameters with patient-specific data;

63. The system of claim 52, further comprising:
predict cardiac response, for use on said subject, using said modified simulated treatment strategy and said disease-specific-calibrated model.

64. A system for determining cardiovascular information of subject, wherein said system comprising:
a memory storing instructions; and
a processor configured to execute the instructions to:
receive patient-specific data;
tune parameters using said patient-specific data; and
generate patient-specific prognostic data from said tuned parameters for use on a said subject.

65. The system of claim 64, wherein said patient-specific prognostic data for said use on said subject causes a user, technician, clinician, or physician to take action on said subject.

66. The system of claim 64, wherein said generated patient-specific prognostic data includes measures of contractility of undamaged myocardium following myocardial infarction, the contractility of individual subregions of the heart in the presence of electrical dyssynchrony, measures of the degree of venoconstriction; and measures of total blood volume and fluid status.

67. The system of claim 64, wherein said generated patient-specific prognostic data includes noninvasive measures of the contractility of myocardium in any disease or condition.

68. The system of claim 64, wherein said processor is further configured to execute the instructions to:
output said generated patient-specific prognostic data.

69. The system of claim 68, wherein said patient-specific prognostic data for said use on said subject causes a user, technician, clinician, or physician to take action on said subject.

70. A computer program product comprising a non-transitory computer readable storage medium containing computer-executable instructions for rapidly predicting cardiac response to a heart condition and treatment strategy of a subject using a compartmental model, said instructions causing a computer to:
receive disease-specific data;
calibrate said compartmental model based on said disease-specific;
receive patient-specific data;
tune parameters using said patient-specific data;
simulate said treatment strategy using said tuned parameters with patient-specific data; and
predict cardiac response, for use on said subject, using said simulated treatment strategy and said disease-specific-calibrated model.

71. The computer program product of claim 70, wherein said processor is further configured to execute the instructions to:
output said predicted cardiac response for said use on said subject.

72. The computer program product of claim 71, wherein said use on said subject of said predicted cardiac response causes a user, technician, clinician, or physician to take action on said subject based on said simulated treatment strategy.

73. The computer program product of claim 70, wherein said patient-specific data includes at least one or any combination of the following:
hemodynamic data; anatomic or functional imaging data from MRI, ultrasound, CT, PET, nuclear or other imaging modalities; ECG, inverse ECG, electroanatomic mapping, or any other cardiac electrical data; medical history; current and past medications; or any other patient-specific information that could affect predictions of heart responses.

74. The computer program product of claim 70, wherein said patient-specific data is acquired from an acquisition device.

75. The computer program product of claim 74, wherein said acquisition device is an image acquisition device.

76. The computer program product of claim 75, wherein said image acquisition device includes at least one or more of any combination of the following:
magnetic resonance imaging (MRI), ultrasound, computed tomography (CT), positron emission tomography (PET), electroanatomic mapping device, or nuclear imaging.

77. The computer program product of claim 70, wherein said acquisition device is a diagnostic device.

78. The computer program product of claim 77, wherein said diagnostic acquisition device includes at least one or more of any combination of the following:
electrocardiogram (ECG or EKG) or other cardiac electrical data device.

79. The computer program product of claim 70, wherein said cardiac response includes at least one or any combination of the following:
changes in heart dimensions, mass, or cavity volumes including growth, hypertrophy, remodeling, shrinkage, or atrophy; changes in heart composition including fibrosis; and changes in heart function including improved or diminished ejection fraction, stroke work, contractility, valvular regurgitation, and synchrony or dyssnchrony of contraction.

80. The computer program product of claim 70, wherein said processor is further configured to execute the instructions to:
evaluate said predicted cardiac response to said simulated treatment strategy.

81. The computer program product of claim 80, wherein said processor is further configured to execute the instructions to:
output said evaluated predicted cardiac response for said use on said subject.

82. The computer program product of claim 81, wherein said use on said subject of said predicted cardiac response causes a user, technician, clinician, or physician to take action on said subject based on said simulated treatment strategy.

83. The computer program product of claim 80, wherein said processor is further configured to execute the instructions to:
modify said simulated treatment strategy based on said evaluated predicted cardiac response.

84. The computer program product of claim 83, wherein said processor is further configured to execute the instructions to:
simulate said modified simulated treatment strategy using said tuned parameters with patient-specific data;

85. The computer program product of claim 84, further comprising:
predict cardiac response, for use on said subject, using said modified simulated treatment strategy and said disease-specific-calibrated model.

86. The computer program product of claim 70, wherein said compartmental model comprises systemic and pulmonary circulations that are represented as a system of resistors and capacitors.

87. The computer program product of claim 86, wherein said compartmental model comprises chambers of the heart that are represented using analytic equations that relate pressure and volume to stress and strain.

88. The computer program product of claim 70, wherein said compartmental model comprises chambers of the heart that are represented as:
spheres or assemblies of multiple spheres; or
substantially spherical shapes or assemblies of multiple substantially spherical shapes.

89. The computer program product of claim 88, wherein said compartmental model further comprises systemic and pulmonary circulations that are represented as a system of resistors and capacitors.

90. The computer program product of claim 88, wherein said compartmental model comprises chambers of the heart that are represented using analytic equations that relate pressure and volume to stress and strain.

91. The computer program product of claim 70, wherein said compartmental model comprises chambers of the heart that are represented using analytic equations that relate pressure and volume to stress and strain.

92. The computer program product of claim 70, wherein said processor is further configured to execute the instructions to:
generate patient-specific prognostic data from said tuned parameters.

93. The computer program product of claim 92, wherein said processor is further configured to execute the instructions to:
output said generated patient-specific prognostic data.

94. The computer program product of claim 93, wherein said patient-specific prognostic data for said use on said subject causes a user, technician, clinician, or physician to take action on said subject.

95. The computer program product of claim 92, wherein said processor is further configured to execute the instructions to:
evaluate said predicted cardiac response to said simulated treatment strategy.

96. The computer program product of claim 95, wherein said processor is further configured to execute the instructions to:
output said evaluated predicted cardiac response for said use on said subject.

97. The computer program product of claim 96, wherein said use on said subject of said predicted cardiac response causes a user, technician, clinician, or physician to take action on said subject based on said simulated treatment strategy.

98. The computer program product of claim 95, wherein said processor is further configured to execute the instructions to:
modify said simulated treatment strategy based on said evaluated predicted cardiac response.

99. The computer program product of claim 98, wherein said processor is further configured to execute the instructions to:
simulate said modified simulated treatment strategy using said tuned parameters with patient-specific data;

100. The computer program product of claim 99, further comprising:
predict cardiac response, for use on said subject, using said modified simulated treatment strategy and said disease-specific-calibrated model.

101. A computer program product comprising a non-transitory computer readable storage medium containing computer-executable instructions for determining cardiovascular information of subject, said instructions causing a computer to:
receive patient-specific data;
tune parameters using said patient-specific data; and
generate patient-specific prognostic data from said tuned parameters for use on a said subject.

102. The computer program product of claim 101, wherein said patient-specific prognostic data for said use on said subject causes a user, technician, clinician, or physician to take action on said subject.

103. The computer program product of claim 101, wherein said generated patient-specific prognostic data includes measures of contractility of undamaged myocardium following myocardial infarction, the contractility of individual subregions of the heart in the presence of electrical dyssynchrony, measures of the degree of venoconstriction; and
measures of total blood volume and fluid status.

104. The computer program product of claim 101, wherein said generated patient-specific prognostic data includes noninvasive measures of the contractility of myocardium in any disease or condition.

105. The computer program product of claim 101, wherein said processor is further configured to execute the instructions to:
output said generated patient-specific prognostic data.

106. The computer program product of claim 105, wherein said patient-specific prognostic data for said use on said subject causes a user, technician, clinician, or physician to take action on said subject.
